(19) United States
(12) Patent Application Publication
(10) Pub. No.: US 2026/0335398 A1
(43) Pub. Date: Oct. 29, 2026

(54) METHODS FOR TREATING PANCREATIC CANCER WITH A COMBINATION OF DES-METHYL PATEAMINE A AND A CLASS I HDAC INHIBITOR

(71) Applicants: Baylor University, Waco, TX (US); The Trustees of Columbia University In The City Of New York, New york, NY (US)

(72) Inventors: Daniel Romo, Waco, TX (US); Kenneth G. Hull, Waco, TX (US); Haoran Xue, Waco, TX (US); Mingzhao Zhu, Waco, TX (US); Susan E. Bates, New York, NY (US); Maryam Safari, New York, NY (US); Antonio Fojo, New York, NY (US)

(73) Assignees: Baylor University, Waco, TX (US); The Trustees of Columbia University In The City Of New York, New york, NY (US)

(21) Appl. No.: 19/490,318

(22) PCT Filed: Jun. 5, 2024
(86) PCT No.: PCT/US2024/032627

§ 371 Date: Dec. 4, 2025

Related U.S. Application Data
(60) Provisional application No. 63/506,481, filed on Jun. 6, 2023.

(51) Int. Cl. A61K 38/15; A61K 31/429; A61K 31/4406; A61P 35/00; G01N 33/575; G01N 33/57525

(52) U.S. Cl. CPC A61K 38/15; A61K 31/429; A61K 31/4406; A61P 35/00; G01N 33/57525; G01N 33/57575

(57) ABSTRACT

A method for treating pancreatic cancer, comprising administering to a subject in need thereof a therapeutically effective amount DMPatA, or a pharmaceutically acceptable salt thereof, and a therapeutically effective amount Class 1 HDAC inhibitor selected from the group consisting of romidepsin, entinostat, and chidamide.

### CROSS-REFERENCE TO RELATED APPLICATION

This application claims the benefit of Provisional Patent Application No. 63/506,481, filed Jun. 6, 2023, which is hereby incorporated by reference.

### STATEMENT REGARDING SEQUENCE LISTING

The Sequence Listing XML associated with this application is provided in XML format and is hereby incorporated by reference into the specification. The name of the XML file containing the sequence listing is 1211-P10WO_Seq_List_20240522.xml. The XML file is 6,376 bytes; was created on May 22, 2024; and is being submitted electronically via Patent Center with the filing of the specification.

### BACKGROUND

Pancreatic Ductal Adenocarcinoma (PDAC), the most common type of pancreatic cancer, arises from the cells lining the ducts of the pancreas. PDAC is a highly lethal cancer, ranking third in cancer-related deaths in the United States. Factors contributing to the high mortality rates of patients with PDAC include late-stage diagnosis, aggressive tumor biology, and resistance to existing treatment options, highlighting an urgent need for innovative therapeutic strategies.

The highly proliferative and invasive PDAC epithelial cells rely on oncogene-driven transcription and mRNA translation, related to frequently mutated drivers and tumor suppressors including KRAS, TP53, CDKN2A, and SMAD4, which disrupt transcriptional regulatory elements and mRNA translation in favor of malignant proliferation. This raises the question of whether targeting factors involved in transcription and translation could be of value in PDAC, such as MYC, histone deacetylases, or the components of the eukaryotic initiation factor 4F (eIF4F) complex, such as eIF4E or eIF4A.

Histone acetyl transferases (HATs) and histone deacetylases (HDACs) play a crucial role in regulating the levels of histone acetylation that affects gene expression. Dysregulation of acetylation is linked to several cancers including PDAC development and metastasis. HDAC inhibitors (HDACis) increase histone acetylation, chromatin accessibility and transcription of several genes including those that regulate cell growth, apoptosis, or differentiation. These insights have led to the FDA approval of HDACis in cutaneous T-cell lymphomas (CTCL), where persistence of histone acetylation was shown to be associated with better patient outcomes. However, the use of HDACis in solid tumors such as PDAC has not yet resulted in sensitivity or survival advantage.

Tumor cells selectively translate mRNAs crucial for growth, metastasis, and adaptation to the tumor environment. The majority of such mRNAs are translated via the strictly regulated cap-dependent mechanism that recognizes their highly structured 5′-untranslated regions (UTRs) with initiation being the rate limiting step. During the initiation step the eukaryotic initiation factor 4F complex (eIF4F) is formed, critical for assembly of the 80s ribosome. An important subunit of the eIF4F complex is the eukaryotic initiation factor 4A (eIF4A, or DDX2), an RNA helicase essential for unwinding the highly structured 5′ UTRs of cap-dependent mRNAs. In some cancers, alterations in the activity and expression of translation initiation factors such as eIF4A are thought to contribute to tumorigenesis, making these factors attractive therapeutic candidates. While clinical development of mRNA translation inhibitors has been challenged by toxicities at effective doses, at least two eIF4A inhibitors are currently in clinical trials.

Despite the advances in the development of treatments for pancreatic cancer, a need exists for therapeutic agents for the treatment of pancreatic cancer. The present disclosure seeks to fulfill this need and provides further related advantages.

### SUMMARY

In one aspect, the present disclosure provides novel therapeutic combinations of an eIF4A inhibitor (des-methyl pateamine A (DMPatA), or a pharmaceutically acceptable salt thereof) and a Class 1 HDAC inhibitor (e.g., romidepsin, entinostat, chidamide) for use in treating pancreatic cancer.

HDAC inhibitors are classified according to their ability to inhibit particular classes of HDACs, histone deacetylases. Class 1 HDACs include the isoforms HDAC 1, 2, 3, and 8 and are localized to the nucleus; Class IIa HDACs include the isoforms 4, 5, 7, and 9; Class IIb HDACs include the isoforms 6 and 10. Most HDAC inhibitors are pan-HDAC inhibitors meaning they inhibit all of the isoforms. However, some such as entinostat and chidamide are primarily Class I inhibitors. Romidepsin is able to inhibit both Class I and II enzymes. However, the potency against Class I HDACs is proportionally so much greater that for all practical purposes, romidepsin is referred to herein as a Class 1 HDAC inhibitor.

In a related aspect, the present disclosure provides a pharmaceutical composition, comprising DMPatA, or a pharmaceutically acceptable salt thereof, and a Class 1 HDAC inhibitor (e.g., romidepsin, entinostat, chidamide) and a pharmaceutically acceptable carrier for use in treating pancreatic cancer.

In further aspects, the present disclosure provides methods for treating pancreatic cancer, modulating the histone code with persistent H3K9 and H3K27 acetylation, reducing in the cellular acetyl-CoA levels, decreasing cellular levels of c-MYC protein and its targets, and impairing glycolysis and OXPHOS processes in a subject. In these methods, DMPatA, or a pharmaceutically acceptable salt thereof, and a Class 1 HDAC inhibitor (e.g., romidepsin, entinostat, chidamide) are administered in a therapeutically effective amount to a subject in need thereof.

In certain embodiments of the above methods, the pancreatic cancer is pancreatic ductal adenocarcinoma.

In certain embodiments, a combination of romidepsin and DMPatA, or a pharmaceutically acceptable salt thereof, is administered.

In other embodiments, a combination of entinostat and DMPatA, or a pharmaceutically acceptable salt thereof, is administered.

In further embodiments, a combination of chidamide and DMPatA, or a pharmaceutically acceptable salt thereof, is administered.

The present disclosure also provides a combination of DMPatA, or a pharmaceutically acceptable salt thereof, and a Class 1 HDAC inhibitor (e.g., romidepsin, entinostat, chidamide) for use in methods for the preparation of a medication for treating pancreatic cancer, modulating the histone code with persistent H3K9 and H3K27 acetylation, reducing in the cellular acetyl-CoA levels, decreasing cellular levels of c-MYC protein and its targets, and/or impairing glycolysis and OXPHOS processes in a subject.

### DESCRIPTION OF THE DRAWINGS

The foregoing aspects and many of the attendant advantages of this invention will become more readily appreciated as the same become better understood by reference to the following detailed description, when taken in conjunction with the accompanying drawings.

FIG. 1A compares cytotoxicity graphs of the effect of romidepsin alone at increasing doses (circles), DMPatA alone at 2 nM (diamond), and combination of romidepsin with DMPatA at 2 nM (triangles) on PANC-1, PANC 02.13, MIA PaCa-2, Capan-1, KLM, AsPC-1 and BXPC3 cells. Romidepsin treatment in both the single agent and the combination was for 6 hours after which it was washed out and medium replaced with fresh medium without (single agent romidepsin) or with DMPatA for an additional 42 hours (combination).

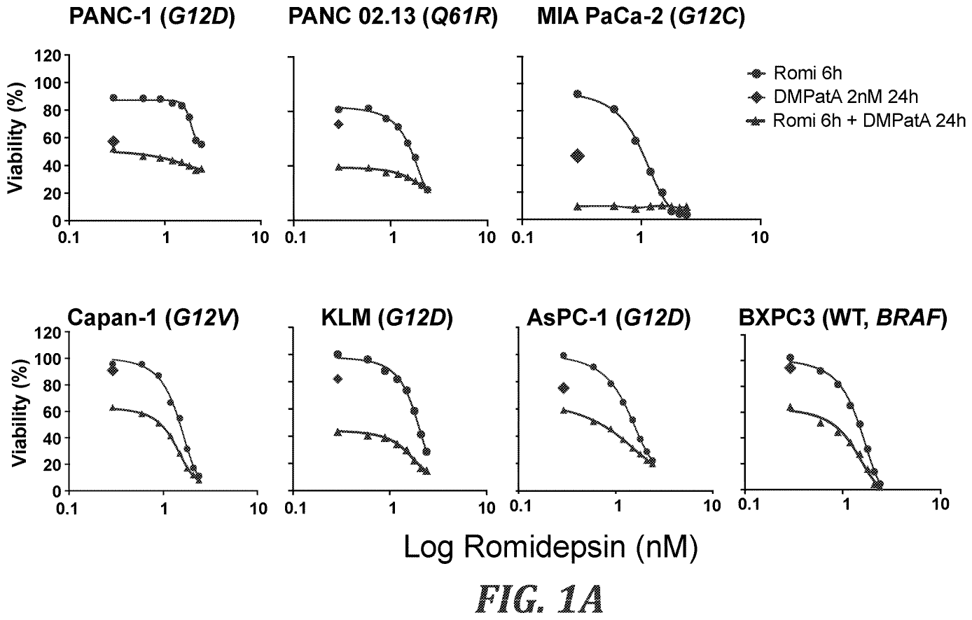

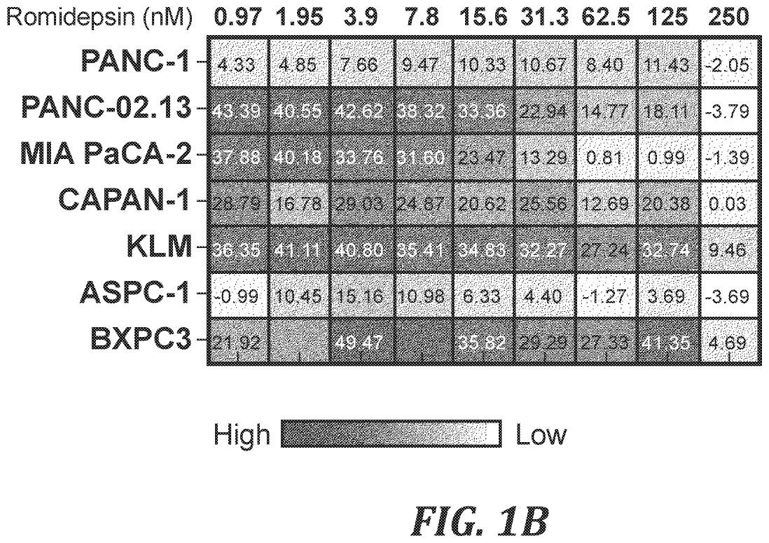

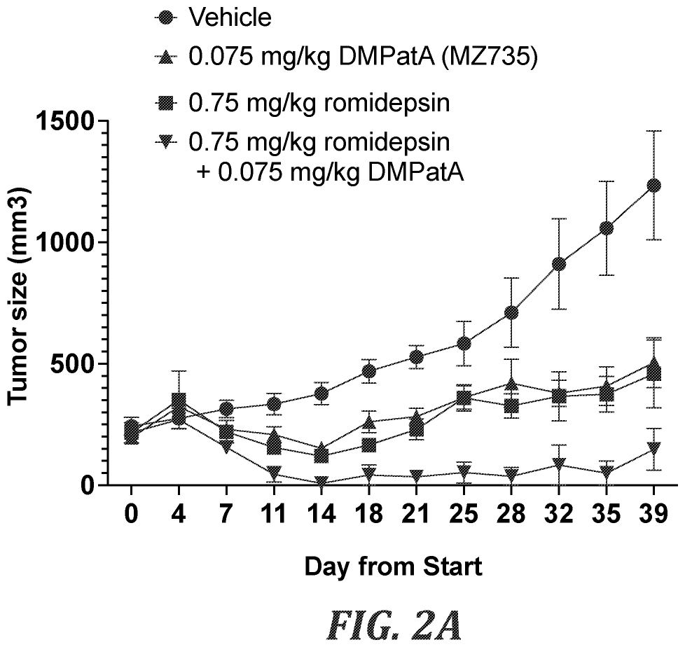

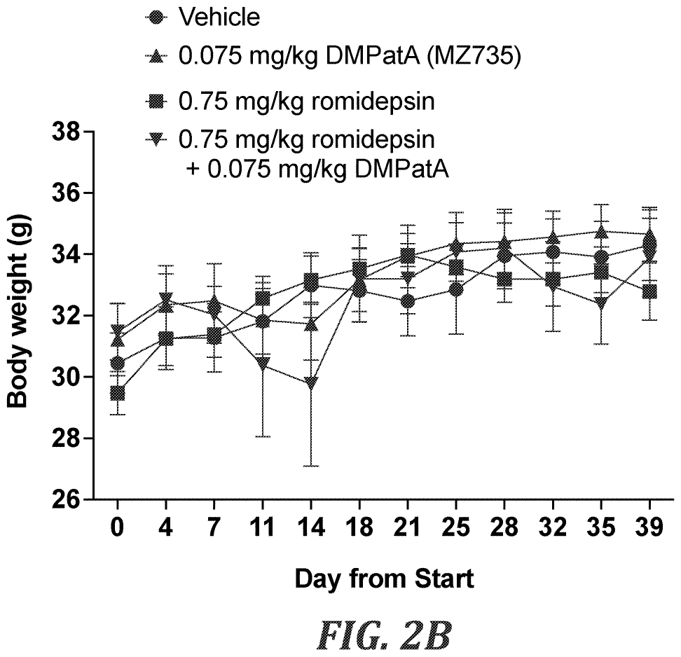

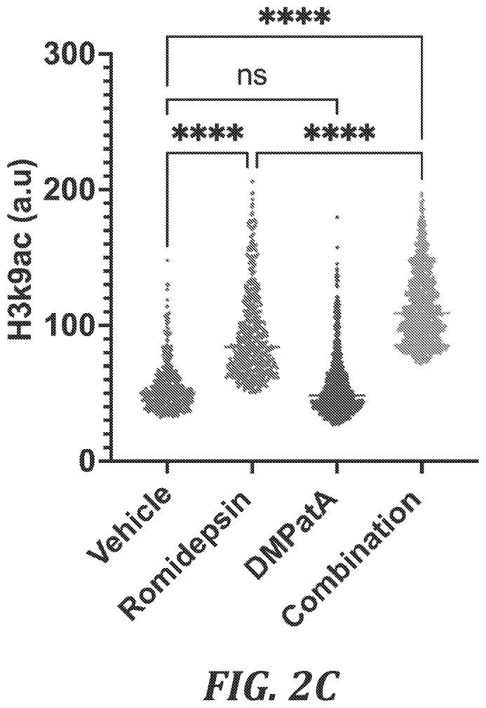

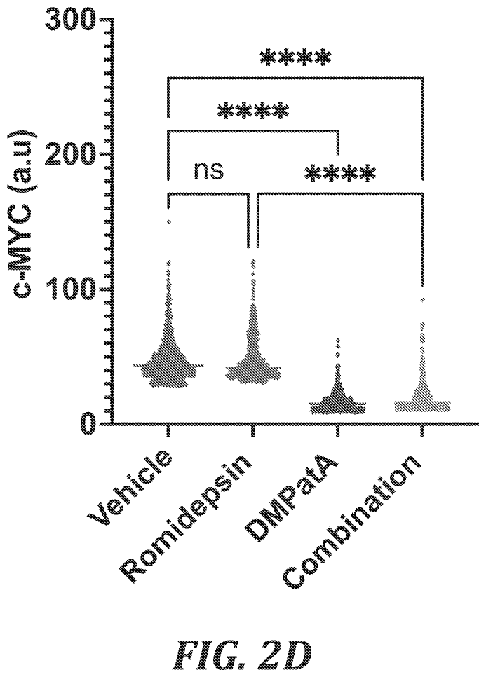

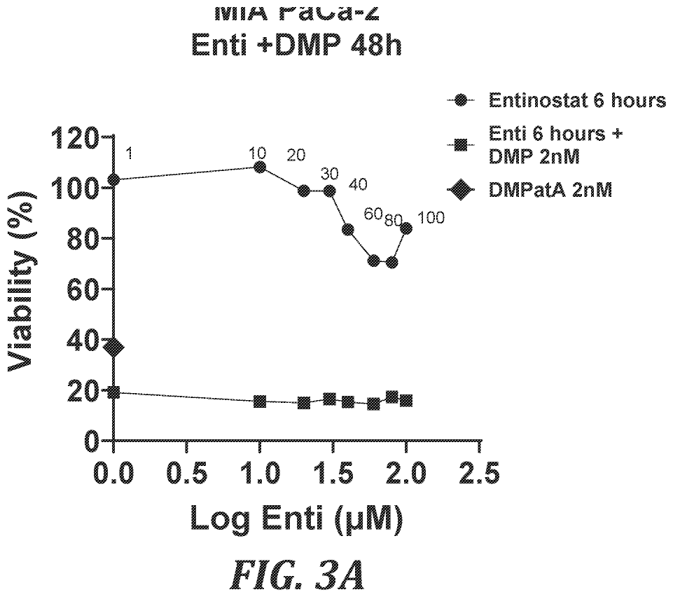

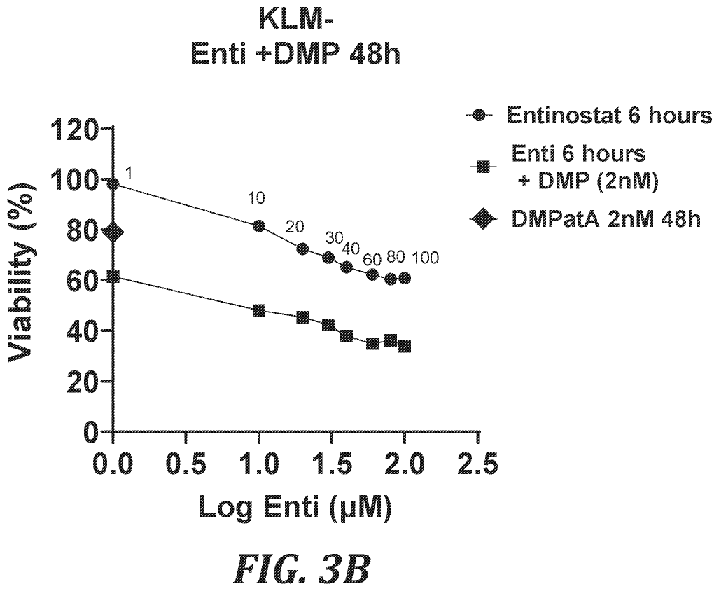

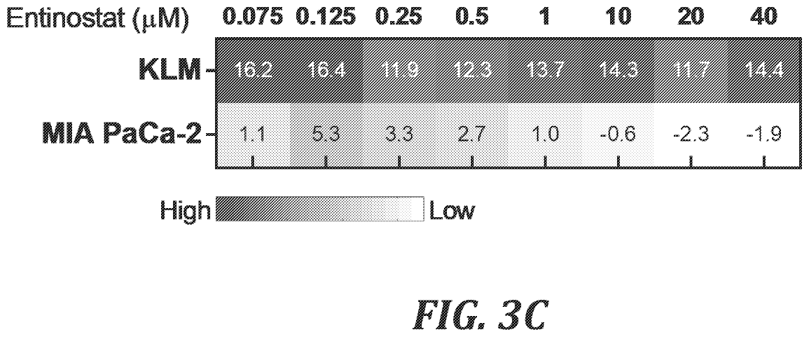

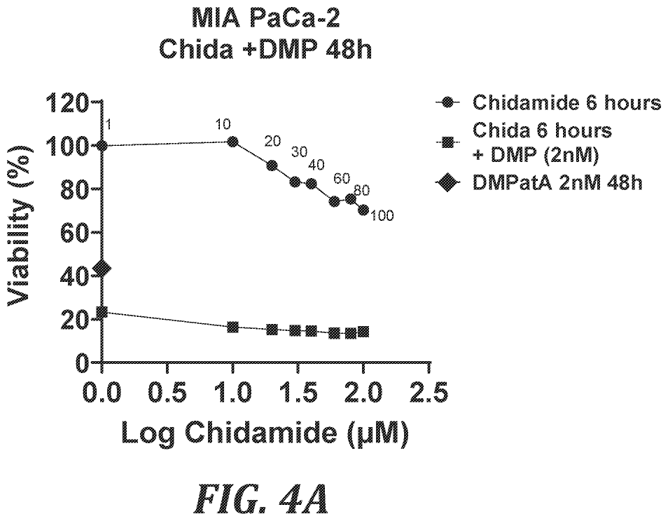

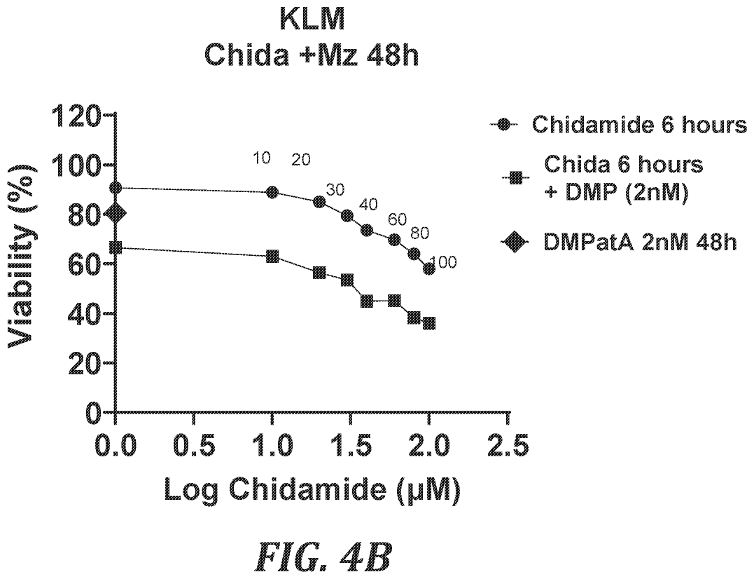

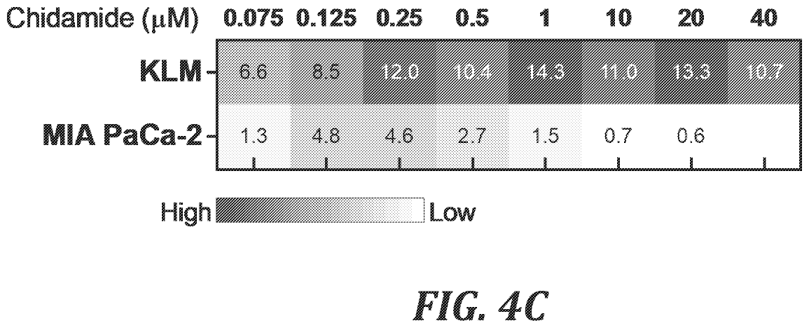

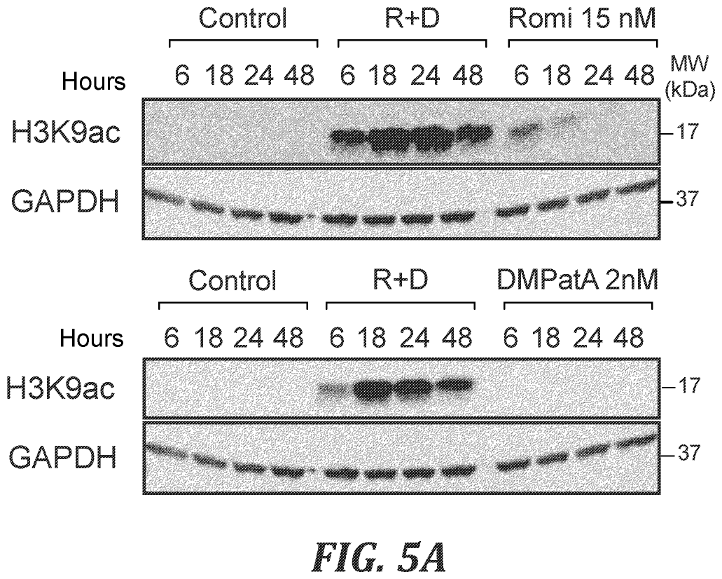

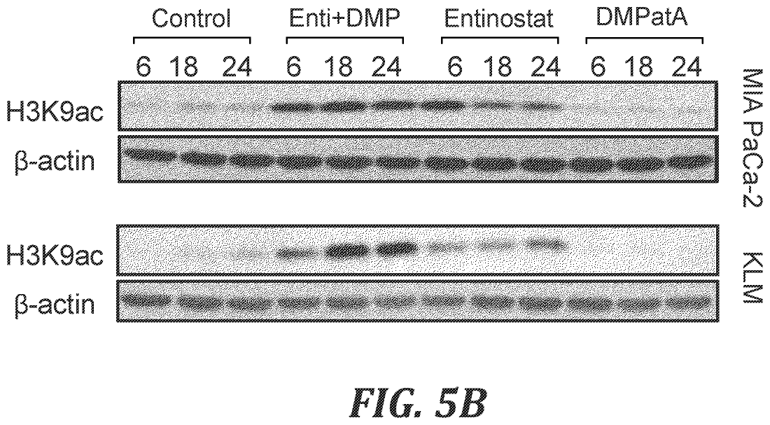

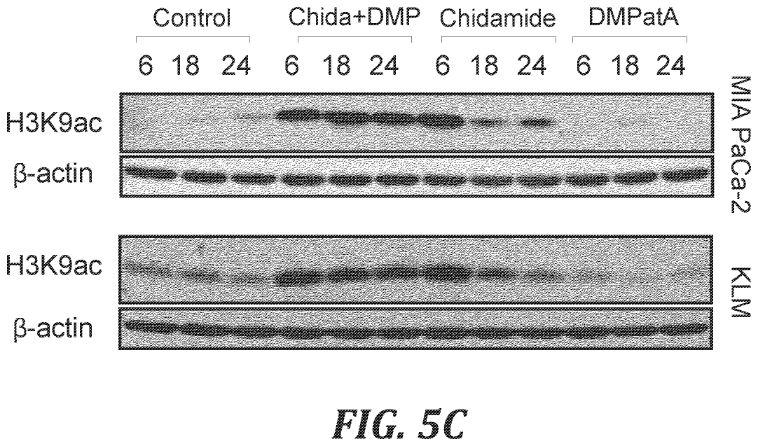

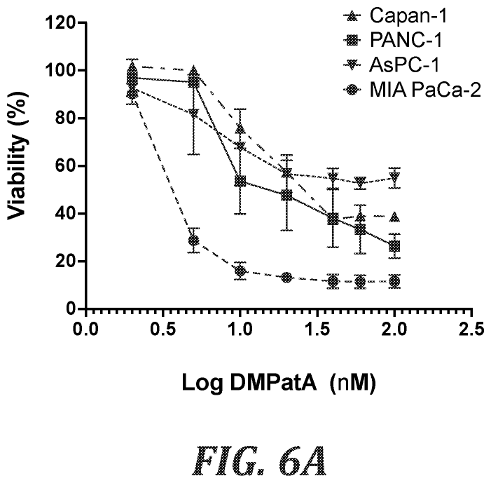

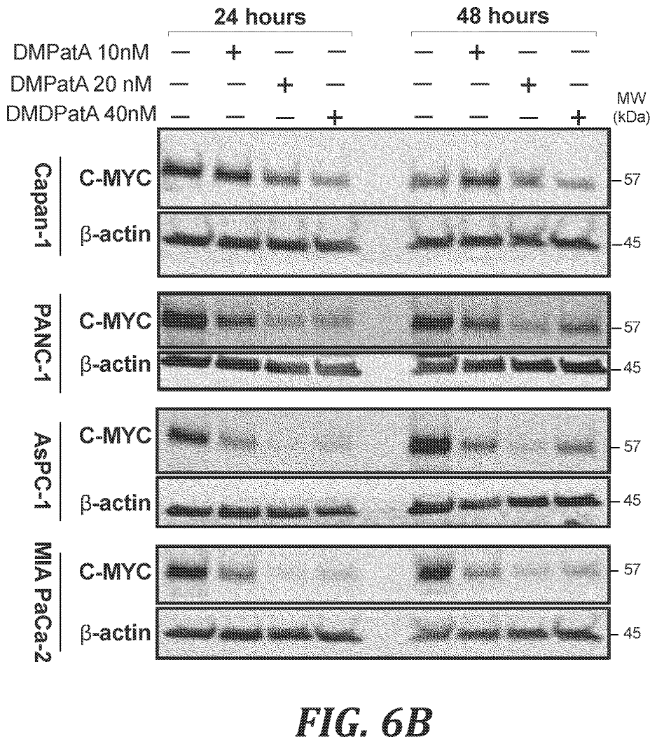

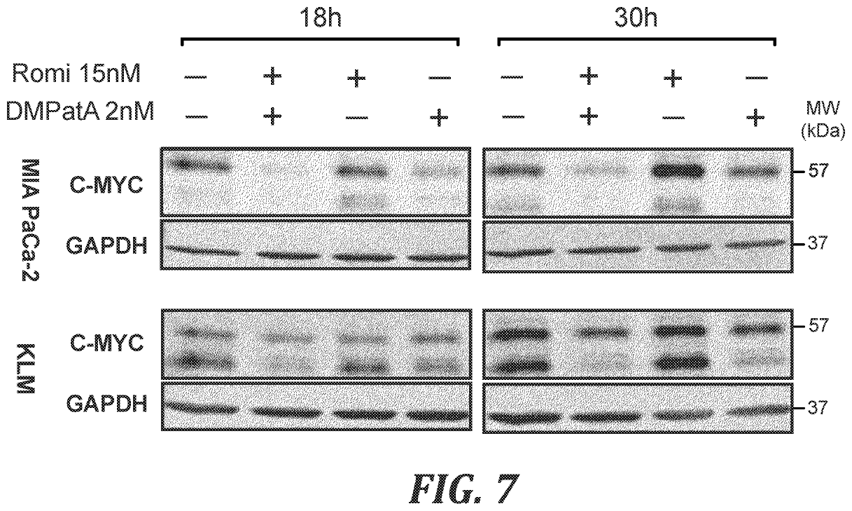

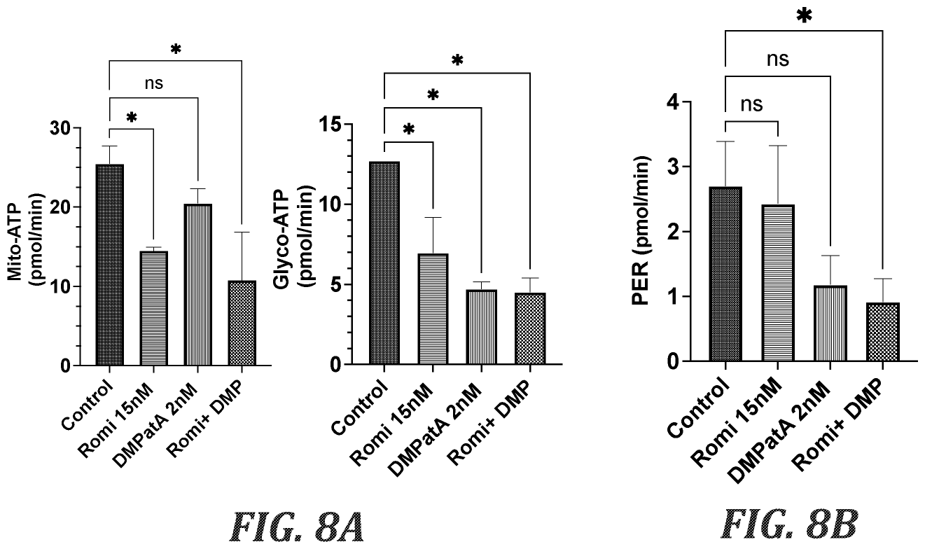

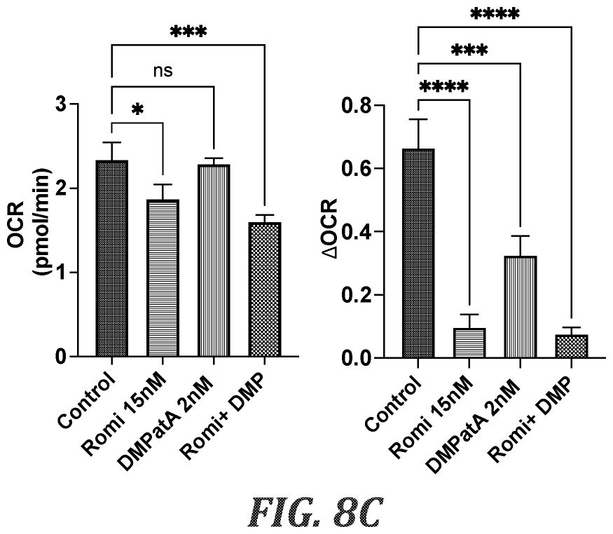

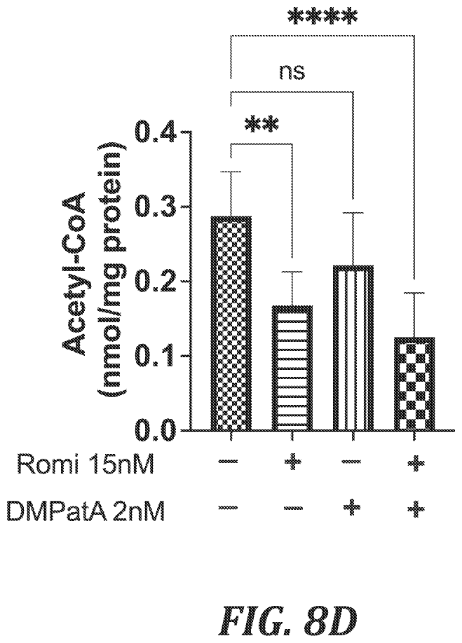

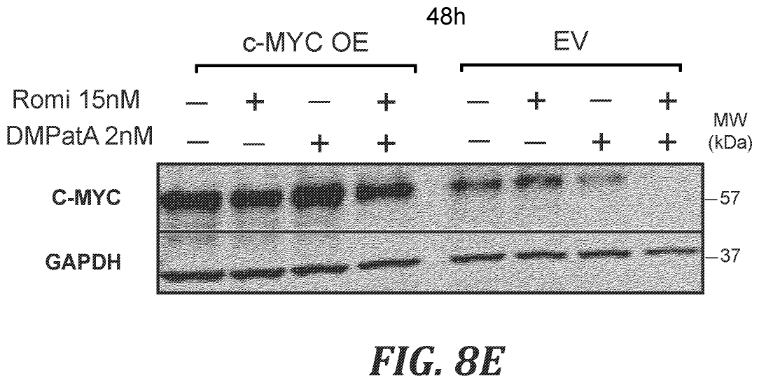

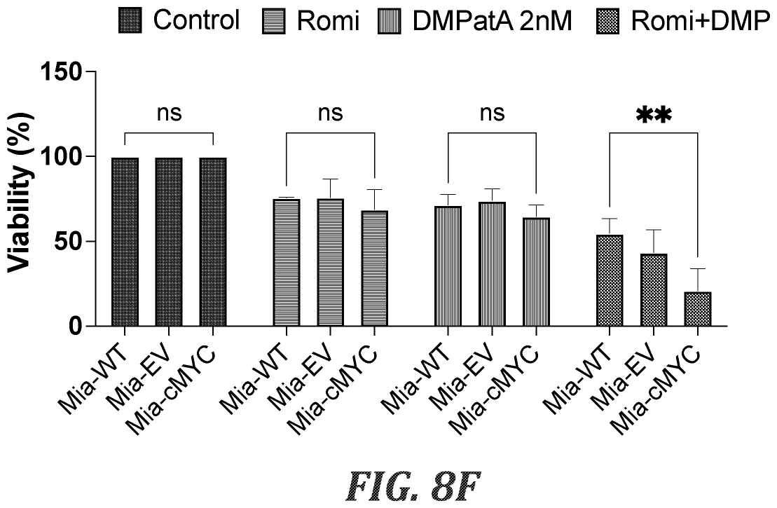

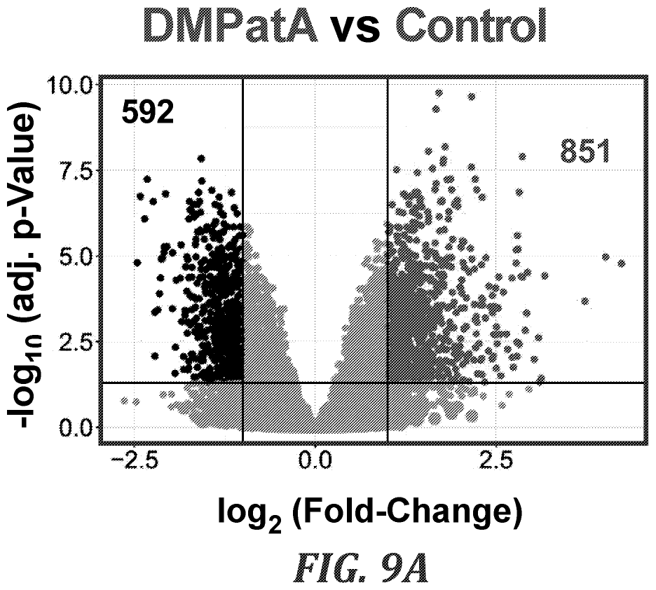

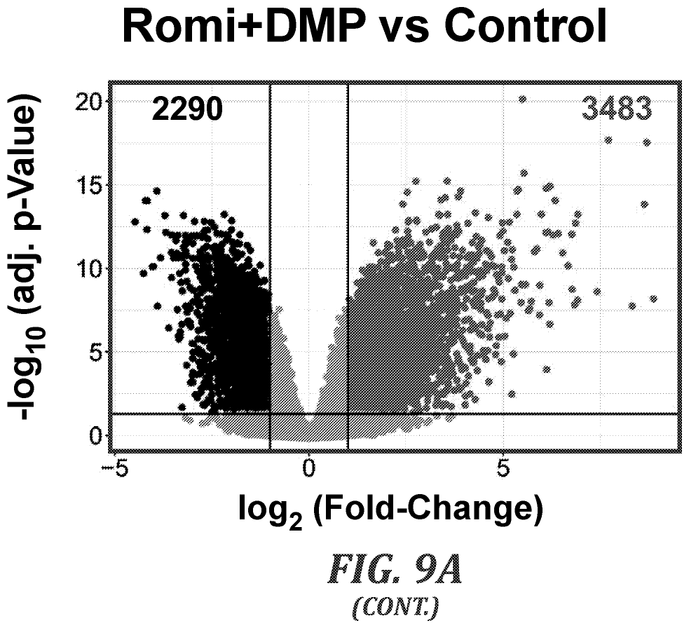

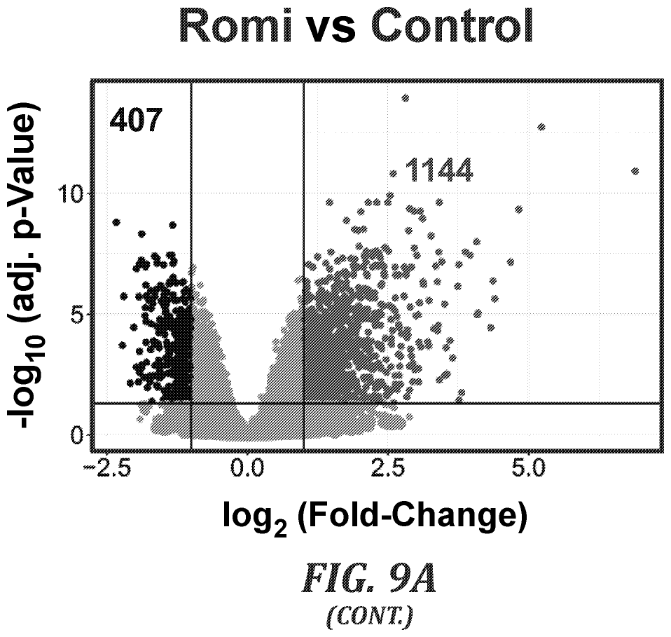

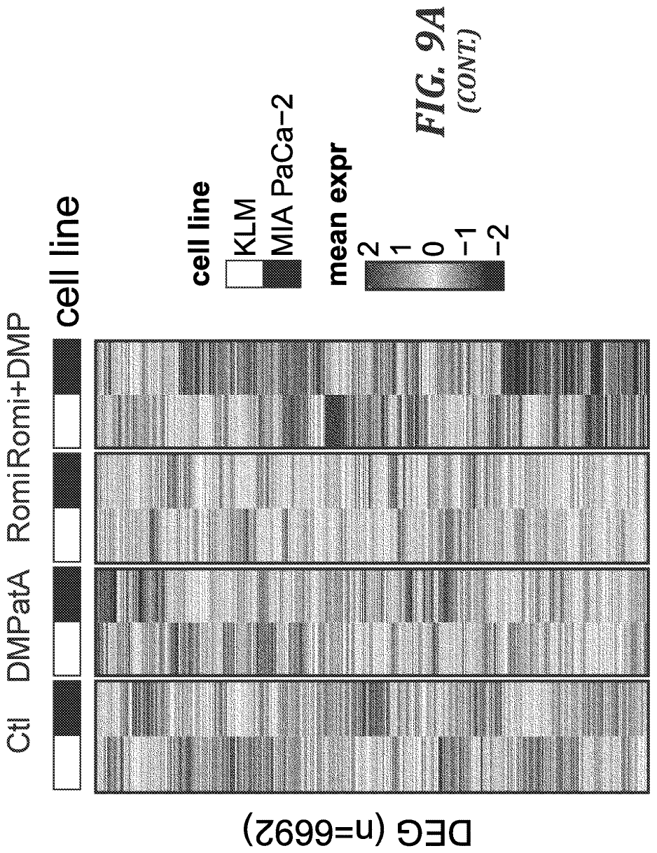

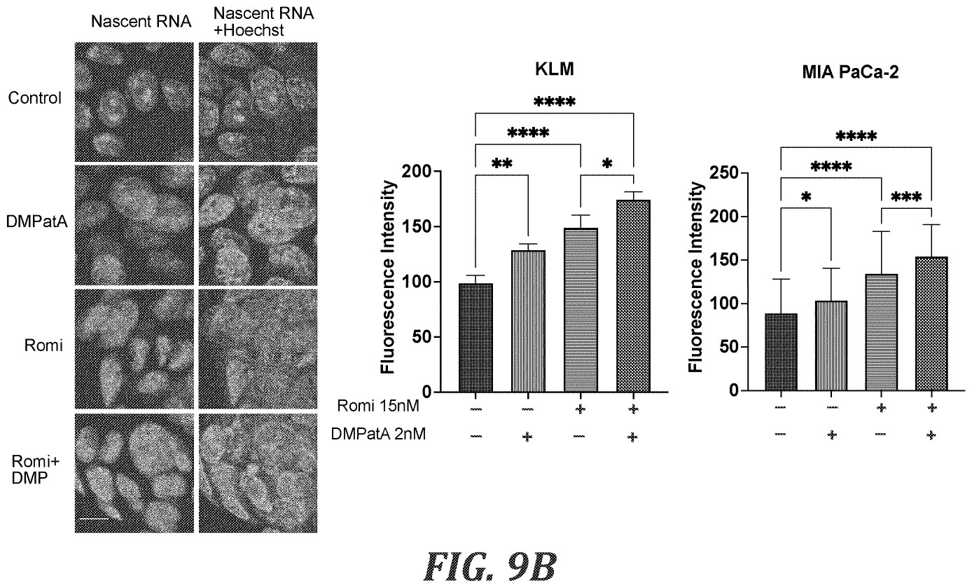

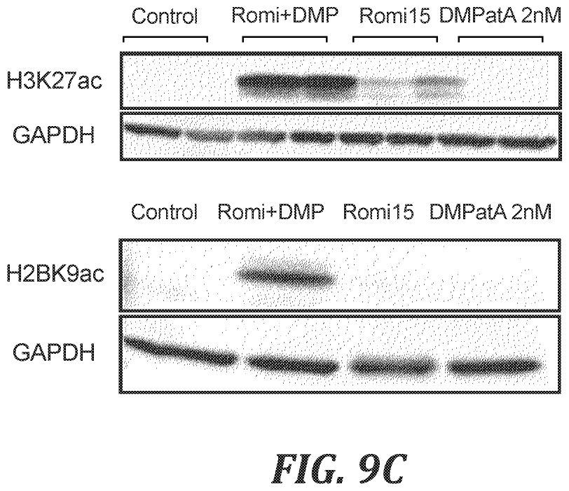

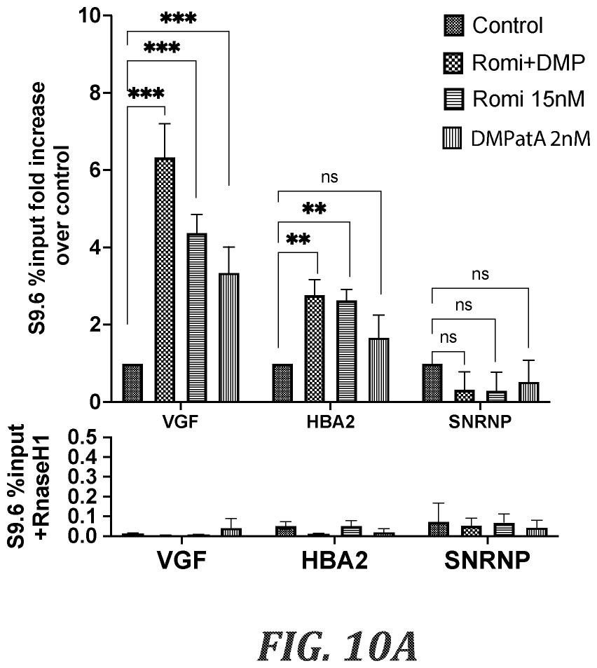

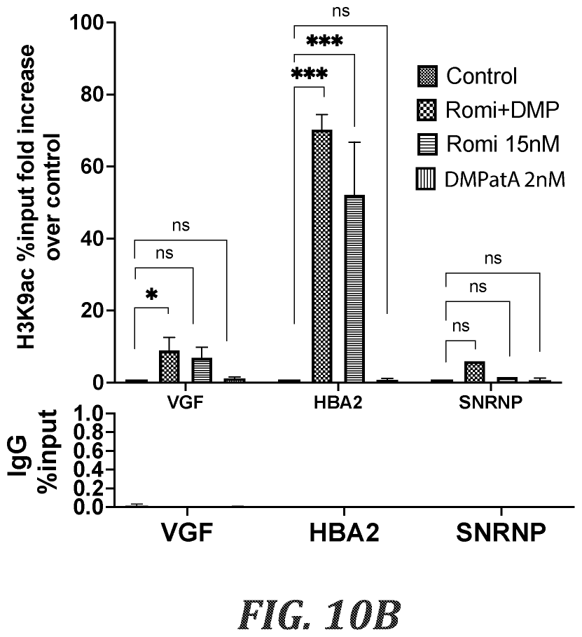

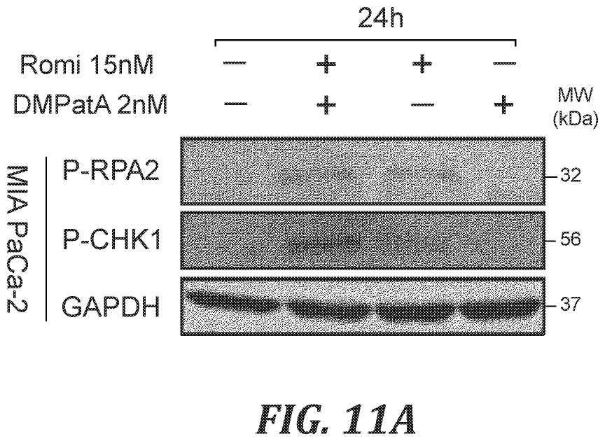

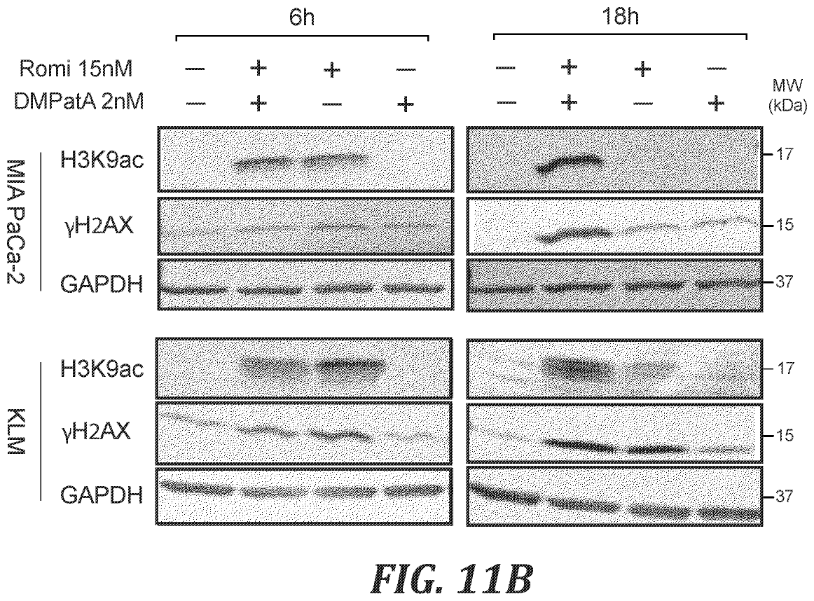

This schedule of romidepsin treatment features a 6-hour exposure followed by washout of romidepsin and replacement with fresh media, whereas most investigators expose cells to 1-2 nM over 48-96 hours of continuous romidepsin. Patients receive romidepsin in a 4-hour infusion and then it is eliminated from circulation with an approximately 3.5 hour half-life. Thus a 6-hour exposure is more clinically relevant. Most cell types are susceptible to romidepsin over non-clinically relevant long treatment durations. Only a subset of cells susceptible to romidepsin when 50 nM and a 6-hour exposure is used. FIG. 1A shows that 42 hr after a 6-hour exposure to only 15 nM romidepsin, remarkable synergy exists in cells also treated with DMPatA. That such a low dose, short exposure to romidepsin in combination with DMPatA demonstrates an apparent and unexpected synthetic lethality.

FIG. 1B shows excess over Bliss synergy (EOB) analysis to quantify the degree of synergy between combining various concentrations of romidepsin with DMPatA at 2 nM.

FIGS. 2A and 2B illustrate the results of a MIA PaCa-2 xenograft model of PDAC in male athymic nude mice. FIG. 2A compares tumor growth (mm³) in mice treated with the indicated concentrations of combination, romidepsin and DMPatA using IP route and vehicles described in the materials and methods section below. FIG. 2B compares body weight (g) of mice treated with the combination and single agents following treatment. FIGS. 2C and 2D compare quantification of H3K9ac and c-MYC antibodies. Frozen sections from MIA PaCa-2 xenograft tumor tissues were subjected to immunohistochemical analysis using antibodies against H3K9ac, and c-MYC. Fluorescence images were captured using fluorescent microscopy and the fluorescent signal from H3K9ac and c-MYC antibodies were quantified. To quantify fluorescence intensity at least 500 cells per condition were measured for each antibody at 40× magnification. The median of the signal intensity is shown. Statistical significance was determined by one-way ANOVA using Tukey multiple comparisons test, ****p value<0.0001.

FIG. 3A compares cytotoxicity graphs of the effect (viability %) of entinostat alone at increasing doses (μM) (circles), DMPatA alone at 2 nM (diamond), and the combination of entinostat with DMPatA at 2 nM (squares) on MIA PaCa-2 cells. Entinostat treatment was for 6 hours after which it was washed out and medium replaced with fresh medium without (single agent entinostat) or with DMPatA for an additional 42 hours (combination).

FIG. 3B compares cytotoxicity graphs of the effect (viability %) of entinostat alone at increasing doses (μM) (circles), DMPatA alone at 2 nM (diamond), and the combination of entinostat with DMPatA at 2 nM (squares) on KLM cells. Entinostat treatment was for 6 hours after which it was washed out and medium replaced with fresh medium without (single agent entinostat) or with DMPatA for an additional 42 hours (combination).

FIG. 3C shows excess over Bliss synergy (EOB) analysis to quantify the degree of synergy between combining various concentrations of entinostat with DMPatA at 2 nM.

FIG. 4A compares cytotoxicity graphs of the effect (viability %) of chidamide alone at increasing doses (μM) (circles), DMPatA alone at 2 nM (diamond), and the combination of chidamide with DMPatA at 2 nM (squares) on MIA PaCa-2 cells. Chidamide treatment was for 6 hours after which it was washed out and medium replaced with fresh medium without (single agent chidamide) or with DMPatA for an additional 42 hours (combination).

FIG. 4B compares cytotoxicity graphs of the effect (viability %) of chidamide alone at increasing doses (μM) (circles), DMPatA alone at 2 nM (diamond), and the combination of chidamide with DMPatA at 2 nM (squares) on KLM cells. Chidamide treatment was for 6 hours after which it was washed out and medium replaced with fresh medium without (single agent chidamide) or with DMPatA for an additional 42 hours (combination).

FIG. 4C shows excess over Bliss synergy (EOB) analysis to quantify the degree of synergy between combining various concentrations of chidamide with DMPatA at 2 nM.

FIGS. 5A-5C are images of immunoblots of protein levels from whole cell extracts of MIA PaCa-2 cell line following treatment with representative combinations Class 1 HDAC inhibitors and DMPatA, DMPatA alone, and HDAC inhibitor alone. FIG. 5A compares immunoblots of H3K9ac (top panel) and GAPDH (bottom panel) protein levels from whole cell extracts of MIA PaCa-2 cells following treatment with the combination of romidepsin and DMPatA and single drugs. Romidepsin treatment was for 6 hours. FIG. 5B compares immunoblots of H3K9ac protein levels from whole cell extracts of MIA PaCa-2 and KLM cells untreated or following treatment with the combination of entinostat and DMPatA and single drugs. Treatments were for various timepoints ranging from 6 to 24 hours. Entinostat treatment in the combination and single drug treatments was only for 6 hours. FIG. 5C compares immunoblots of H3K9ac protein levels from whole cell extracts of MIA PaCa-2 and KLM cells untreated or following treatment with the combination of chidamide and DMPatA and single drugs. Treatments were for various timepoints ranging from 6 to 24 hours. Chidamide treatment in the combination and single drug treatments was only for 6 hours.

FIG. 6A compares cytotoxicity graphs of the effect (viability) of DMPatA on various PDAC cell lines (Capan-1, PANC-1, AsPC-1, MIA PaCa-2) following 48 hours of treatment.

FIG. 6B compares immunoblots of c-MYC protein levels from whole cell extracts of various PDAC cell lines (Capan-1, PANC-t, AsPC-1, MIA PaCa-2) treated with DMPatA (10 nM and 20 nM) and DMDPatA (Pateamine A) (40 nM). Treatment was for 24 and 48 hours.

FIG. 7 compares immunoblots of c-MYC protein levels from whole cell extracts of PDAC cell lines (MIA PaCa-2 and KLM) following treatment of the cells with the combination as well as with DMPatA at 2 nM. Romidepsin treatment was for 6 hours.

FIG. 8A compares quantification of ATP production rates. Seahorse XF Real-Time ATP Rate Assay was performed under control conditions or after treatment with the combination and single agents. Treatment was for a total of 18 h (i.e., 6-hour romidepsin followed by 12-hour washout with 2 nM DMPatA (combo) or without (Romi alone)). FIG. 8A left panel—ATP production rate from mitochondrial respiration (Mito-ATP); FIG. 8A right panel—ATP production rate from glycolysis (Glyco-ATP). At least two biological replicate experiments with n=3 technical replicates were performed. Statistical significance was determined by one-way ANOVA using Tukey multiple comparison's test in the Prism software. *p value<0.01.

FIG. 8B presents the results of a metabolic flux analysis showing basal glycolysis (Proton Efflux Rate-PER) in MIA PaCa-2 cells following treatment with combinations or single drug treatments for a total of 18 h, as mentioned in FIG. 8A. At least two independent biological replicate experiments were performed with n=3 technical replicates and data were expressed as mean±SD. Statistical significance was determined by one-way ANOVA using Tukey multiple comparison's test.

FIG. 8C left panel presents the results of metabolic flux analysis showing basal mitochondrial respiration (OXPHOS) (oxygen consumption rate, OCR) in MIA PaCa-2 cells following treatment with combinations or single drugs. Treatment was for a total of 18 h, as mentioned in FIG. 8A. Statistical significance was determined by one-way ANOVA, ***p value<0.001, *p value<0.01. FIG. 8C right panel presents the results of quantification of glutamine consumption. Cells were treated as mentioned in FIG. 8A. Prior to the metabolic measurements cells were starved of glucose, glutamine, and pyruvate for 1-hour after which glutamine was added and response was recorded as ΔOCR (i.e. OCR before and after glutamine addition). Statistical significance was determined by one-way ANOVA, ****p value<0.0001, ***p value<0.0005.

FIG. 8D compares cellular acetyl-CoA levels measured in MIA PaCa-2 cells using LC-MS following treatment of cells with the combination or with single agents as mentioned in FIG. 8A. At least three biological replicate experiments were carried out. Statistical significance was determined by two-way ANOVA, ****p value<0.0001, ***p value<0.0004.

FIG. 8E compares immunoblots of c-MYC protein levels in whole cell lysate of MIA PaCa-2 cells transfected with MYC over-expressing (MYC OE) or empty vector (EV), with or without treatment with the combination or single agents for a total of 48 hours as mentioned in FIG. 8A. Romidepsin treatment was for only 6 hours.

FIG. 8F compares the results of a cytotoxicity assay (viability %) of cMYC and EV MIA PaCa-2 cells following treatment. One-way ANOVA with Tukey multiple comparison's test used for statistical analysis. **p value<0.002.

FIG. 9A (four panels) shows volcano plots of statistical significance (adjusted p-value) versus fold change, and heatmap of gene expression analysis showing the results of RNA-seq of the cells treated with the combination or with single agents. In volcano plots the most upregulated genes are towards the right, the most downregulated genes are towards the left. Plots show a significant increase in the overall gene expression in the cells treated with the combination. Cells treated for a total of 18 h (i.e., 6-hour romidepsin followed by 12-hour incubation with 2 nM DMPatA (combo) or without (Romi alone)).

FIG. 9B left panel compares EU-labeled nascent RNA and (Hoechst counterstain) in KLM cells treated with combination and single agents. Scale bar: 1 μm. FIG. 9B right panels present quantification of fluorescence intensities of nascent RNA synthesis in both KLM and MIA PaCa-2 cell lines. Statistical significance was determined by one-way ANOVA using Tukey multiple comparison's test, ****p value<0.0001, **p value<0.0002, **p value<0.002.

FIG. 9C compares immunoblots showing levels of the H3K27ac and H2BK9ac in MIA PaCa-2 cells untreated or following treatment with combination and single drugs.

Treatments were for various timepoints ranging from 6 to 48 hours. Romidepsin treatment in combination and single drug treatments was for 6 hours.

FIG. 10A illustrates the fold increase in the percent of input values in DRIP-qPCR for the VGF, HBA2, and SNRPN loci in MIA PaCa-2 cells treated with or without the combination or single agents. Genomic DNA was treated in vitro with or without RNaseH1 before immunoprecipitation using S9.6 antibody. Treatment was for 6+3 hours (i.e., 6-hour romidepsin followed by 3-hour washout with 2 nM DMPatA (combo) or without). Statistical significance was determined by 3-way ANOVA, ***p value<0.001. This experiment confirms the formation of R-loops at the investigated gene loci.

FIG. 10B illustrates ChIP-qPCR fold increase in the percentage of input values for the VGF, HBA2, and SNRPN loci in MIA PaCa-2 cells treated with or without the combination or single agents. Genomic DNA immunoprecipitated using H3K9ac antibody. Treatment was for 6+3 hours as described above. Statistical significance was determined by 3-way ANOVA, ***p value<0.001. This confirms that both histone acetylation and R-loops are present at the same gene loci, consistent with the observation that increased histone acetylation leads to increased R-loop formation.

FIG. 11A compares immunoblots of p-RPA and p-CHK1 protein levels from whole cell extracts following treatment with the combination and romidepsin for a total of 24 h (i.e., romidepsin treatment was for 6 h followed by 18-hour incubation with 2 nM DMPatA (combo) or without (Romi alone)).

FIG. 11B compares immunoblots of H3K9ac and γ-H2AX protein levels from whole cell extracts following treatment with the combination of DMPatA and romidepsin or single agents.

### DETAILED DESCRIPTION

Pancreatic ductal adenocarcinoma (PDAC) is a highly lethal solid tumor, primarily resistant to standard therapies, in need of innovative treatments. Epigenetic changes along with core driver mutations in KRAS, p53, and other genes play a role in tumor evolution and metastasis. Despite success in some malignancies, epigenetic modifiers including HDAC inhibitors (HDACi) have not achieved regulatory approvals in solid tumors.

The results herein describe the effect of novel therapeutic combinations comprising a Class 1 HDAC inhibitor (e.g., romidepsin, entinostat, and chidamide) with an eIF4A inhibitor (i.e., DMPatA) in PDAC cells.

The inventors found that the combination of DMPatA and the Class 1 HDAC inhibitors described herein is effective for treating pancreatic cancer. This combination augments the HDAC inhibitor activity by sustaining the histone acetylation caused by the HDAC inhibitor. The combination treatment enhances the metabolic disruption induced by individual drugs by concomitantly targeting both the glycolytic and oxidative phosphorylation (OXPHOS) pathways. The inventors discovered a synergy between DMPatA and the Class 1 HDAC inhibitors described herein in the treatment of pancreatic cancer.

The interaction between DMPatA and the Class 1 HDAC inhibitors described herein is synergistic, not additive. In an additive interaction, the overall effect is just the sum of the effects of each drug used alone, without any enhanced efficacy from their combination. The Excess over Bliss method is a method commonly used to assess the degree of synergy in a novel drug combination. See FIG. 1B. The Excess over Bliss method is a statistical measure that uses response of a target (e.g., cell viability to each drug) and quantifies the deviation between observed outcomes from combined treatments and those anticipated under the assumption of independence based on the Bliss model, determining synergistic interactions in multifactorial experimental settings. The data from cell viability assays establishes the synergy of the romidepsin and DMPatA combination. See FIG. 1A.

In addition to statistical evidence, visual examination of the combination curves also points to a synergistic interaction. For example, the concentration of romidepsin at 15 nM is noted as inducing minimal to no toxicity on its own. It is only the addition of 2 nM DMPatA (itself a low concentration) that results in lethality. This is also observed in the immunoblot analyses that show only a small effect of 15 nM romidepsin on histone acetylation after 6 hr, a finding that is rapidly reversed. See FIG. 5A (top panel). Addition of DMPatA induces persistent and far greater histone acetylation than would ever be expected from an agent that has no known interaction with HDACs and no ability to induce histone acetylation on its own. This histone hyperacetylation is responsible for the excess cell death observed after exposure to the drug combination.

The third line of evidence supporting a synergistic interaction of the combination is from animal studies. In murine tumors obtained 72 hours after the last drug treatment, histone hyperacetylation is readily detected by immunochemistry analysis. See FIGS. 2C and 2D. This remarkable result stands in contrast to studies carried out in humans showing histone acetylation at 4 hours after infusion starts in the peripheral blood cells of all patients, persisting to 24 hours only in a small subset. Those patients tended to have a better clinical response. Both gene expression and histone acetylation were highly reversible within 24 hours in patients. In these murine tumors, histone acetylation was clearly detectable at elevated levels at 72 hours.

Treatment with the representative romidepsin/DMPatA combination increased metabolic stress and induced cell death in PDAC cell lines with diverse KRAS/BRAF mutations, while also prolonging histone acetylation compared to a low dose, short exposure to romidepsin alone. The elevated histone hyperacetylation caused hyper-transcription, accumulation of co-transcriptional R-loops, induction of replication stress and double-stranded DNA damage in PDAC cells. Using a MIA PaCa-2 xenograft model, in vivo experiments showed suppression of tumor growth upon treatment with the romidepsin/DMPatA combination.

HDAC inhibitors, initially discovered for their anti-tumor ability by promoting tumor cell differentiation were eventually recognized to target HDAC enzymes causing acetylation of histones and other non-histone proteins. Hyperacetylation exploits vulnerabilities of tumor cells promoting tumor specific cell death attributed to activation of genes regulating differentiation and apoptosis, and generation of ROS and DNA damage. Additionally, they promote cell cycle arrest by inducing hyperacetylation at the promoter of genes such as CDKN1A, leading to increased p21^{WAF1/CIP1} expression. These findings underscore the clinical relevance of HDAC inhibitors but also highlight their challenges in treating solid tumors and suggest their potential use in combination therapies to enhance their effectiveness.

Studies on eIF4A inhibitors have shown that a subset of proteins involved in cell cycle regulation, proliferation, and apoptosis including MYC, CYCLIND1, and BCL2 rely on cap-translation, and inhibition of cap-dependent translation induces apoptosis. Although various tumor types are sensitive to inhibition of protein translation, toxicities have hindered development of these agents, encouraging their development at lower doses in combination regimens.

As described herein, previous findings on the effects of HDAC and eIF4A inhibitors in monotherapy were validated, including a decrease in c-MYC levels. It was believed that this combination would induce metabolic stress though a combination of reduced c-MYC levels with both compounds and reduction of acetyl-CoA levels due to the HDAC inhibitor. Combining the known downregulation of c-MYC and acetyl-CoA by an HDAC inhibitor with the inhibition of c-MYC by an eIF4A inhibitor was hypothesized to induce metabolic stress. As described herein, using non-toxic concentrations of both agents confirmed metabolic stress and demonstrated marked synergy in PDAC cell lines with concentrations that are clearly subtherapeutic and nontoxic alone. The extent of the synergy was completely unexpected and suggested that the underlying mechanism was not the c-MYC based additive mechanism noted above. Rather, it is now apparent that persistent, enforced histone hyperacetylation mediates synergistic activity. Not only is histone hyperacetylation thought to create metabolic stress, but it also means that chromatin is persistently open and available for gene transcription. Conducting gene expression profiling showed an increase in global gene expression with a combination of both drugs, confirmed by elevated levels of nascent RNA, indicating hypertranscription.

Induction of gene expression following histone hyperacetylation was evaluated. Hyper-transcription, a concept from stem cell biology, refers to an increase in nascent RNA production beyond normal cellular levels. Oncogene-induced hyper-transcription is linked to R-loop accumulation, heightened transcription-replication conflicts, replication stress, and DNA damage. Hence, targeting oncogene-induced replication stress has emerged as a promising therapeutic approach. HDAC enzymes play a role in maintaining replication fork progression. Inhibition or loss of HDAC1/2 and 3 has been shown to slow the replication fork speed and increases replication stress. While the role of HDACs in maintaining replication fork stress is established, the impact of histone acetylation-induced hyper-transcription, resulting from inhibition of these enzymes, in inducing replication stress has not been thoroughly investigated. The data demonstrate that an HDAC/translation inhibitor combination treatment described herein induces hyper-transcription, leading to replication stress, supporting this phenomenon. Importantly, the combination results in a higher degree of hyperacetylation, replication stress, and double-stranded DNA damage compared to the single agent HDAC inhibitor, romidepsin. Furthermore, the data highlights the involvement of both HDACs and eIF4A/cap-translation in preventing transcription-replication conflicts, for which a better understanding can have important therapeutic implications.

RNA-seq analysis of cells treated with the combination or with single-agent DMPatA found mitochondrial respiration and apoptotic signaling related genes among the most impacted. Additionally, genes directly involved in pathways that regulate histone modifications are among the significantly enriched pathways in cells treated with the combination and DMPatA alone. This suggests that the translation of these regulatory proteins is cap-dependent and provides insights into how DMPatA may contribute to sustaining the hyperacetylation initiated by HDAC inhibitors.

MYC overexpression was shown to further sensitize the cells to the combinations. MYC activation occurs in 43% of advanced PDAC cases and is associated with more aggressive tumor behavior. In cancer cells with elevated c-MYC levels, c-MYC enhances transcription of genes throughout the entire genome and is not limited to “MYC target genes. It does so by accumulating in the promoters of active genes and increasing transcription. It is therefore likely that treatment of the cells overexpressing MYC with the combination augments the hyper-transcription phenotype caused by persistent histone hyperacetylation, enhancing replication stress and DNA damage in these cells.

The effect of combining a different eIF4A inhibitor, zotatifin, with romidepsin, as well as combining other Class 1 HDAC inhibitors, such as entinostat and chidamide, with DMPatA, was investigated. In the former, zotatifin demonstrated reduced potency, compared to DMPatA, in PDAC cells both alone and in the combination. However, the latter indicated that a potential common Class-I HDAC regulatory mechanism is targeted when these HDAC inhibitors are combined with DMPatA.

In one aspect, the present disclosure provides novel therapeutic combinations of an eIF4A inhibitor (des-methyl pateamine A (DMPatA), or a pharmaceutically acceptable salt thereof) and a Class 1 HDAC inhibitor (e.g., romidepsin, entinostat, chidamide) for use in treating pancreatic cancer. These drug combinations show significant antitumor activity in both in vitro and in vivo models of pancreatic cancer.

In a related aspect, the present disclosure provides a pharmaceutical composition, comprising DMPatA, or a pharmaceutically acceptable salt thereof, and a Class 1 HDAC inhibitor (e.g., romidepsin, entinostat, chidamide) and a pharmaceutically acceptable carrier for use in treating pancreatic cancer.

In further aspects, the present disclosure provides methods for treating pancreatic cancer, modulating the histone code with persistent H3K9 and H3K27 acetylation, reducing in the cellular acetyl-CoA levels, decreasing cellular levels of c-MYC protein and its targets, and impairing glycolysis and OXPHOS processes in a subject. In these methods, DMPatA, or a pharmaceutically acceptable salt thereof, and a Class 1 HDAC inhibitor (e.g., romidepsin, entinostat, chidamide) is administered in a therapeutically effective amount to a subject in need thereof.

MYC overexpression and activation occurs in 43% of advanced PDAC cases and is associated with a more aggressive tumor behavior, poor prognosis, and resistance to conventional therapies. Involvement of MYC in key oncogenic pathways, such as KRAS signaling, and its role in maintaining the aggressive phenotype of PDAC make it an important target for therapeutic strategies. Therefore, treatment strategies that downregulate MYC such as the DMPatA/HDAC inhibitor combination have important therapeutic potential.

The data described herein show that MYC overexpression further sensitizes PDAC cells to the combination treatment, suggesting that the overexpression of MYC with its downstream metabolic derangements can render pancreatic cancer cells more vulnerable to the DMPatA/HDAC inhibitor combination. Thus, MYC could serve as a biomarker for identifying patients who could particularly benefit from the DMPatA/Class 1 HDAC combinations.

The data described herein suggests stratifying patients by cancer cell MYC expression in early clinical trials to determine whether MYC could enrich the patient population likely to benefit. If efficacy of the combination was markedly greater in the presence of MYC overexpression, then MYC could be developed as a companion diagnostic, as recommended in FDA guidelines, to aid in the selection of the DMPatA/Class 1 HDAC combination for individual patients, thus developing a precision therapy for pancreatic cancer.

Therefore, in a further aspect, based on the data set forth herein, the disclosure supports the use of MYC as a biomarker for pancreatic cancer, for example, by identifying patients with pancreatic cancer overexpressing MYC who thus would benefit from treatment with the DMPatA/Class 1 HDAC combinations.

As noted above, the present disclosure provides compositions and methods that include DMPatA, or a pharmaceutically acceptable salt thereof. The chemical structures of pateamine A (PatA), DMDAPatA, and DMPatA are shown below.

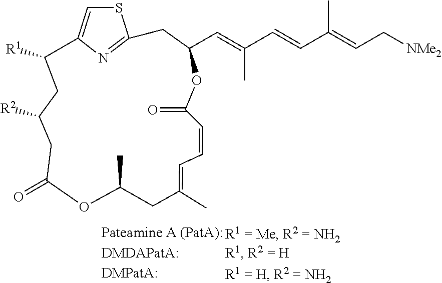

DMPatA can be prepared as described in U.S. Pat. No. 10,889,596.

Pharmaceutically acceptable salts of DMPatA may be formed from DMPatA and a pharmaceutically acceptable organic acid (e.g., carboxylic acid) or inorganic acid (e.g., mineral acid). Representative acids include hydrochloric acid, sulfuric acid, phosphoric acid, formic acid, acetic acid, trifluoroacetic acid, maleic acid, fumaric acid, succinic acid, tartaric acid, oxalic acid, citric acid, malic acid, benzoic acid, toluenesulfonic acid, methanesulfonic acid, and benzenesulfonic acid. Such salts may be formed during or after the synthesis of DMPatA.

A representative pharmaceutically acceptable salt of DMPatA is a formate.

DMPatA, or a pharmaceutically acceptable salt thereof, and the Class 1 HDAC inhibitor (e.g., romidepsin, entinostat, chidamide) may be formulated together or separately in a pharmaceutically acceptable carrier to provide a pharmaceutical composition for administration. The pharmaceutical composition may be formulated as a solid for oral administration or as an injectable solution for intravenous administration (e.g., saline or dextrose for injection).

Suitable carriers include those suitable for administration to an animal (e.g., a human subject). Pharmaceutical compositions suitable for injectable use include sterile aqueous solutions (e.g., saline, dextrose) and dispersions.

The compounds and compositions described herein can be administered parenterally (e.g., (dosed) intravenously, intraperitoneally, or orally). The compounds and compositions described herein can be orally administered, for example, with an inert diluent or carrier, enclosed in hard- or soft-shell gelatin capsule, or compressed into tablets. For oral therapeutic administration, the compounds and compositions can be combined with excipients and used in the form of ingestible tablets, buccal tables, troches, capsules, elixirs, suspensions, syrups, wafers, and the like. The amount of active compounds in such therapeutically useful compositions is such that a suitable dosage is obtained.

In another aspect, the disclosure provides methods for treating pancreatic cancer using the novel therapeutic combinations of DMPatA, or a pharmaceutically acceptable salt thereof, and a Class 1 HDAC inhibitor (e.g., romidepsin, entinostat, chidamide) as described herein. In the methods, therapeutically effective amounts of DMPatA, or a pharmaceutically acceptable salt thereof, and the Class 1 HDAC inhibitor are administered to a subject in need thereof.

In certain embodiments, the pancreatic cancer is pancreatic ductal adenocarcinoma (PDAC). In certain of these embodiments, the pancreatic cancer is a MYC-overexpressing/MYC-addicted PDAC tumor.

In related aspects, the disclosure provides methods for modulating the histone code with persistent H3K9 and H3K27 acetylation, reducing in the cellular acetyl-CoA levels, decreasing cellular levels of c-MYC protein and its targets, and impairing glycolysis and OXPHOS processes in a subject that includes administering to a subject in need thereof amounts of DMPatA, or a pharmaceutically acceptable salt thereof, and the Class 1 HDAC inhibitor effective to achieve these effects.

In the methods, the term “therapeutically effective amount” refers to an amount effective, at dosages and for periods of time necessary, to achieve the desired therapeutic result, such as reduced levels of the protein products of MYC, CCND1, or CD44 genes. A therapeutically effective amount of a compound may vary according to factors such as the disease state, age, sex, and weight of the subject, and the ability of the compound to elicit a desired response in the subject. Dosage regimens can be adjusted to provide the optimum therapeutic response. A therapeutically effective amount is also one in which any toxic or detrimental effects of the administered compound are outweighed by the therapeutically beneficial effects.

It is noted that dosage values can vary with the severity of the condition to be alleviated. For any particular subject, specific dosage regimens can be adjusted over time according to the individual need and the professional judgment of the person administering or supervising the administration of the compositions. Dosage ranges are typically selected by a medical practitioner. The amount of active compound in the composition can vary according to factors such as the disease state, age, sex, and weight of the subject. Dosage regimens can be adjusted to provide the optimum therapeutic response. For example, a single bolus can be administered, several divided doses can be administered over time or the dose can be proportionally reduced or increased as indicated by the exigencies of the therapeutic situation.

In the methods, the administration of the compound can be systemic administration to the subject. The term “subject” includes mammalian organisms. Examples of subjects include humans and non-human mammals. In certain embodiments, the subject is a human.

The terms “administering,” “contacting,” or “treating” include any method of delivery of DMPatA, or a pharmaceutically acceptable salt thereof, and the Class 1 HDAC inhibitor into a subject's system.

In certain embodiments of the above methods, the HDAC inhibitor described herein and DMPatA, or a pharmaceutically acceptable salt thereof, are administered at the same time. In certain of these embodiments, the HDAC inhibitor and DMPatA are administered in a single composition. In other of these embodiments, the HDAC inhibitor and DMPatA are administered separately in individual compositions. In other embodiments of the above methods, the HDAC inhibitor and DMPatA are administered sequentially at different times.

In certain embodiments of the methods, a combination of romidepsin and DMPatA, or a pharmaceutically acceptable salt thereof, is administered. The effectiveness of the combination of romidepsin and DMPatA for treating pancreatic cancer is shown in FIGS. 1A, 1B, 2A, 2B, 2C, and 2D. FIG. 1A compares cytotoxicity graphs of the effect of romidepsin alone at increasing doses (circles), DMPatA alone at 2 nM (diamond), and combination of romidepsin with DMPatA at 2 nM (triangles) on PANC-1, PANC 02.13, MIA PaCa-2, Capan-1, KLM, AsPC-1 and BXPC3 cells. FIGS. 2A and 2B illustrate the results of a MIA PaCa-2 xenograft model of PDAC in male athymic nude mice. FIG. 2A compares tumor growth (mm³) in mice treated with the indicated concentrations of combination, romidepsin and DMPatA using intraperitoneal (IP) route and vehicles described in the materials and methods section. FIG. 2B compares body weight (g) of mice treated with the combination and single agents following treatment. FIGS. 2C and 2D compare quantification of H3K9ac and c-MYC antibodies in MIA PaCa-2 xenograft tumor tissues subjected to immunohistochemical analysis using antibodies against H3K9ac, and c-MYC. The data demonstrate the effectiveness of the combination of romidepsin and DMPatA for treating pancreatic cancer.

In other embodiments of the methods, a combination of entinostat and DMPatA, or a pharmaceutically acceptable salt thereof, is administered. The effectiveness of the combination of entinostat and DMPatA for treating pancreatic cancer is shown in FIGS. 3A and 3B. FIG. 3A compares cytotoxicity graphs of the effect (viability %) of entinostat alone at increasing doses (μM) (circles), DMPatA alone at 2 nM (diamond), and combination of entinostat with DMPatA at 2 nM (squares) on MIA PaCa-2 cells. FIG. 3B compares cytotoxicity graphs of the effect (viability %) of entinostat alone at increasing doses (μM) (circles), DMPatA alone at 2 nM (diamond), and the combination of entinostat with DMPatA at 2 nM (squares) on KLM cells. The data demonstrate the effectiveness of the combination of entinostat and DMPatA for treating pancreatic cancer. FIG. 3C shows excess over Bliss synergy (EOB) analysis to quantify the degree of synergy between combining various concentrations of entinostat with DMPatA at 2 nM.

In further embodiments of the methods, a combination of chidamide and DMPatA, or a pharmaceutically acceptable salt thereof, is administered. The effectiveness of the combination of chidamide and DMPatA for treating pancreatic cancer is shown in FIGS. 4A and 4B. FIG. 4A compares cytotoxicity graphs of the effect (viability %) of chidamide alone at increasing doses (μM) (circles), DMPatA alone at 2 nM (diamond), and the combination of chidamide with DMPatA at 2 nM (squares) on MIA PaCa-2 cells. FIG. 4B compares cytotoxicity graphs of the effect (viability %) of chidamide alone at increasing doses (μM) (circles), DMPatA alone at 2 nM (diamond), and the combination of chidamide with DMPatA at 2 nM (squares) on KLM cells. The data demonstrate the effectiveness of the combination of chidamide and DMPatA for treating pancreatic cancer. FIG. 4C shows excess over Bliss synergy (EOB) analysis to quantify the degree of synergy between combining various concentrations of entinostat with DMPatA at 2 nM.

Increased histone acetylation was also observed at H3K9ac modification (FIG. 5A). A drug class effect is suggested by the similar results observed with the combinations of DMPatA with other HDAC inhibitors, including entinostat and chidamide (FIGS. 5B and 5C, respectively).

FIGS. 5A-5C are images of immunoblots of protein levels from whole cell extracts of MIA PaCa-2 cell line following treatment with representative combinations Class 1 HDAC inhibitors and DMPatA, DMPatA alone, and HDAC inhibitor alone. FIG. 5A compares immunoblots of H2BK9ac protein levels from whole cell extracts of MIA PaCa-2 cells following treatment with the combination of romidepsin and DMPatA and single drugs. FIG. 5B compares immunoblots of H3K9ac protein levels from whole cell extracts of MIA PaCa-2 and KLM cells untreated or following treatment with the combination of entinostat and DMPatA and single drugs. FIG. 5C compares immunoblots of H3K9ac protein levels from whole cell extracts of MIA PaCa-2 and KLM cells untreated or following treatment with the combination of chidamide and DMPatA and single drugs.

Histone hyperacetylation plays a significant role in regulating gene expression. In pancreatic cancer key tumor suppressor genes like CDKN2A/p16, TP53, and SMAD4/DPC4 are epigenetically silenced, disrupting normal cell cycle control and apoptosis regulation that contributes to tumor development and drug resistance. HDAC inhibitors can counteract this by increasing histone acetylation, thereby maintaining an open chromatin state that promotes gene expression particularly of genes that promote cell differentiation. At low doses, HDAC inhibitors are known to promote a differentiated or more mature phenotype in cells. As one example, leukemic cells can be induced to make hemoglobin after low dose HDAC inhibition. However, at higher doses HDAC inhibitors induce cell death in many cell types, and this is associated with a marked degree of acetylation—hyperacetylation. Hyperacetylation exploits vulnerabilities of tumor cells promoting tumor specific cell death, inducing hypertranscription and DNA damage along with metabolic stress. Although HDAC inhibitors have shown efficacy in hematological malignancies, their effectiveness in solid tumors like pancreatic cancer remains limited. In hematological malignancies such as CTCL, the persistence of histone acetylation was shown to be associated with better patient outcomes. The significance of this combination therapy is that it remarkably augments and prolongs histone hyperacetylation, inhibits cancer cell proliferation and showcases the potential of epigenetic therapy in treating pancreatic cancer. HDAC inhibition results in histone acetylation due to unrestrained histone acetyltransferase activity. Notably, the HDAC inhibitor/DMPatA inhibitor combination provokes persistently increased levels of histone acetylation, termed hyperacetylation. Before the studies that are the subject of this application, histone acetylation was understood to be the initial, direct effect of HDAC inhibition, with secondary mechanisms leading to cell death. Numerous secondary mechanisms were invoked to explain the effects on both cell differentiation and cell death; induction of genes regulating differentiation; induction of genes directing cell cycle arrest and apoptosis; DNA damage; alterations in the mitotic spindle causing cell cycle arrest; and increased acetylation of cellular proteins, particularly those chaperone proteins needed to stabilize oncoproteins. As described herein, histone hyperacetylation resulting from the combination of romidepsin and eIF4A inhibition is shown to be more directly involved in the cellular events that follow, including R-loop formation, hypertranscription, and depletion of acetyl CoA, hindering pancreatic cancer cell metabolism.

eiF4A Inhibitor DMPatA Prevents PDAC Cell Proliferation and Reduces MYC Protein Levels.

Previous studies have reported that inhibitors of translation initiation prevent cell proliferation and reduce levels of critical oncoproteins such as MYC and cyclinD1. The effects of DMPatA, a novel inhibitor of eIF4A helicase, was evaluated on four PDAC cell lines. All cells were sensitive to treatment with IC50s ranging from 2.8 to 10 nM (FIG. 6A). As expected, treatment with both DMPatA and DMDAPatA (des-methyl des-amino pateamine A, an earlier analogue of DMPatA) reduced c-MYC levels in all cell lines. However, DMPatA achieved comparable effects at lower doses. The efficacy of another eIF4Ai, zotatifin (eFT226), was evaluated on MIA PaCa-2 and KLM cells. The data indicated that DMPatA exhibits significantly greater potency in suppressing proliferation of cells at lower doses compared to zotafitin.

Combining the HDAC-Inhibitor Romidepsin with the eIF4A Inhibitor, DMPatA, Results in Synergistic Cell Death at Low Drug Concentrations.

It was initially hypothesized that the HDAC-inhibitor romidepsin could cause synergistic cell death when combined with DMPatA via a reduction in MYC levels previously seen with both eIF4A inhibitors and with romidepsin in KRAS cells. As shown in FIG. 1A, the effects of the combination on cell proliferation in seven PDAC cell lines with different KRAS or BRAF mutations was evaluated: PANC-1 (G12D), PANC 02.13 (Q61R), MIA PaCa-2 (G12C), Capan-1 (G12V), KLM (G12D), ASPC1 (G12D), and BXPC3 (Kras WT, BRAF). Romidepsin treatment was only for 6 hours as a clinically relevant exposure duration, after which it was washed out and medium replaced with fresh medium that was either drug-free (single agent romidepsin) or maintained 2 nM of DMPatA for an additional 42 hours (combination). For DMPatA treatment as a single agent control cells were incubated with 2 nM of DMPatA for 48 hours.

As shown in FIG. 1A, all the tested cell lines were sensitive to the combination (triangles), albeit to varying degrees, with MiaPaCa-2 cells most sensitive to both DMPatA and combination treatments. Excess over Bliss analysis confirmed synergy at low concentrations of romidepsin with DMPatA at 2 nM (FIG. 1B). Additionally, the synergistic effect of combining romidepsin with zotatifin (at higher doses of 4 and 10 nM) was assessed on MIA PaCa-2 and KLM cells was assessed. The data showed that similar to the observation in monotherapy, the extent of synergy was significantly diminished with zotatifin when compared to the combination of romidepsin with only 2 nM DMPatA.

Because treatment with DMPatA decreased c-MYC protein levels, MYC protein levels were assessed after treatment with low doses of both DMPatA (2 nM) and romidepsin (15 nM) and reduced protein levels of c-MYC following treatment of cells with the combination as well as with DMPatA was found (FIG. 7). Because c-MYC is known to alter/reprogram cellular metabolism to support oncogenesis, the effect of the combination on oxidative phosphorylation and glycolysis was examined.

### Combination Treatment Impaired Metabolism, and MYC Overexpression Sensitizes Cells to the Treatment.

Because reprograming/augmentation of cellular metabolism by c-MYC is known to support oncogenesis, the Seahorse XF analyzer was used to assess the impact of the treatments on c-MYC-driven metabolism including ATP production, glycolysis, and mitochondrial oxidative phosphorylation (OXPHOS). Extracellular acidification rates, also known as proton efflux rate (PER), were measured for glycolysis and mitochondrial oxygen consumption rates (OCR) for OXPHOS. We utilized the 6 h exposure followed by 18 h washout strategy for romidepsin described earlier. Treatment with romidepsin alone and the combination reduced both mitochondrial and glycolytic ATP production (Mito- and Glyco-ATP), while DMPatA treatment mainly impaired the production of ATP by glycolysis (Glyco-ATP) (FIG. 8A). This was confirmed by separately assessing glycolysis (FIG. 8B) and mitochondrial function (OCR) (FIG. 8C left panel). In MIA PaCa-2 cells, DMPatA impaired glycolysis, romidepsin impaired mitochondrial function and the combination treatment impaired both glycolysis and the mitochondria. Additionally, to assess the impact of treatment on glutamine oxidation, a primary energy source for cancer cells, especially those driven by c-MYC, the ability of glutamine to rescue mitochondrial energy production was examined in cells starved for glucose, glutamine, and pyruvate (FIG. 8C, right panel). In control cells, glutamine addition increased basal levels of oxygen consumption (ΔOCR), rescuing mitochondrial oxidative phosphorylation by providing glutamine as the energy source. However, treatment with romidepsin alone or the combination and to a lesser extent DMPatA, impaired glutamine rescue of mitochondrial function.

Previous studies have demonstrated that histone acetylation following treatment with the HDAC inhibitor romidepsin reduces cellular acetyl-CoA levels, a key donor for histone acetylation and an important metabolic intermediate. These studies suggested that reduced acetyl-CoA levels might underlie the impaired glutamine utilization. Measuring cellular acetyl-CoA levels showed a significant reduction in cells treated with either romidepsin alone or the combination in both MIA PaCa-2 cell line (FIG. 8D).

To study the contribution of MYC reduction to the metabolic impairment and cell death following treatment with the combination, MYC overexpression was forced in MIA PaCa-2 cells (FIG. 8E). The MYC expressing cells were more sensitive to treatment with the combination (FIG. 8F), suggesting MYC-overexpressing/MYC-addicted PDAC cells may be more sensitive to the combination treatment.

### Combination Treatment Increased Gene Expression.

To gain further insight into the effects of the combination treatment gene expression was profiled in MIA PaCa-2 and KLM cells treated with vehicle, the single agents, or the combination for a total of 18 h (i.e., 6-hour romidepsin treatment followed by 12-hour washout with 2 nM DMPatA or without), probing whether alterations observed following combination treatment could be explained by changes in gene expression. Initial analysis showed a significant increase in the number of differentially expressed (DEG) genes in cells treated with the combination compared to the single agents (5773 DEGs for combination treatment versus 1551 for romidepsin and 1443 for DMPatA) (FIG. 9A).

It should be noted that altered expression due to HDAC inhibition also includes reduction of gene expression, and the volcano plots demonstrate the marked increase in numbers of genes that are repressed. The field to date has not been able to explain the mechanism by which gene expression is reduced after HDAC inhibition. One possibility is that transcription factors are removed from some genes and redirected toward the areas of hyperacetylated open chromatin. As described herein, in addition to hypertranscription, there are also a far greater number of genes that are repressed with the combination than seen with single agent HDAC inhibition.

Thus, an examination of the gene expression profiles induced by the combination suggest that it is, as seen with greater and longer duration histone acetylation, the same types of gene expression patterns seen in many studies of HDAC inhibitors. Gene expression is increased to a far greater extent than with monotherapy, described below as hypertranscription, and gene repression also exceeds that observed in monotherapy.

### Combination Treatment Induced Hyperacetylation and Hypertranscription.

A well-known effect of HDAC inhibition is altered gene expression, thought to be due to reduced chromatin compaction following increased histone acetylation with both gene induction and gene repression being demonstrated. Given the short exposure (6 h) and relatively low dose (15 nM) of romidepsin, single agent romidepsin treatment has only a modest increase in gene expression. However, the combination was associated with a dramatic increase in the number of genes with altered expression levels. This apparent hypertranscription observed with the combination, was confirmed by the increase in nascent RNA output above the level of normal cells, following treatment for a total of 18 hours (i.e., 6-hour romidepsin followed by 12-hour washout with or without 2 nM DMPatA), quantified using incorporation of the modified RNA precursor 5-EU. In both KLM and MIA PaCa-2 cells treatment with the combination significantly increased mRNA synthesis (FIG. 9B). An increase in RNA synthesis was also seen only with romidepsin and to a lower extent with DMPatA. Because HDAC inhibitors cause histone hyperacetylation and having observed the increase in gene expression following treatment with the combination, the impact of the combination on histone acetylation was assessed. The addition of DMPatA led to a remarkable persistence of increased H3K9 acetylation well above that observed with romidepsin alone (again at a low dose (15 nM) and duration (6 hr) of exposure) (FIG. 9C top panel). DMPatA alone did not result in histone acetylation but sustained the increased histone acetylation initiated by romidepsin (FIG. 9C bottom panel). Increased histone acetylation was also observed with other histone markers such as H3K27ac and H2BK9ac modifications (FIG. 9C). A drug class effect is suggested by the similar results observed with combinations of DMPatA with other HDAC inhibitors, including entinostat and chidamide (FIGS. 5B and 5C, respectively).

### Combination Treatment Induces Accumulation of Co-Transcriptional R-Loops.

Romidepsin-induced histone hyperacetylation leads to the accumulation of R-loops that can increase genome instability. Because treatment of cells with the combination increased global transcription and led to persistence of histone hyperacetylation, it was postulated the treatment could similarly enhance the formation of R-loops and contribute to cell death. R-loop accumulation was further confirmed using DRIP-qPCR. To choose candidate genes to test for R-loop accumulation by DRIP-qPCR the R-Loop DB and the R-loopBase databases were used and identified amongst the genes whose expression increased, those with R-loop forming sequences (RLFS), computationally predicted by integrating experimental RNA-DNA hybrid data. After choosing two prospective gene candidates—VGF and HBA2—DRIP-analysis was performed in MIA PaCa-2 cells using the S9.6 antibody to immunoprecipitate genomic DNA with or without RNaseH1 treatment, followed by qPCR and confirmed a significant increase in the percent of input of these loci in the samples treated with the combination as well as with romidepsin alone (FIG. 10A) consistent with accumulation of co-transcriptional R-loops. Increased R-loop accumulation was also observed in the cells treated with single agent DMPatA. SNRNP as a negative locus was also examined in MIA PaCa-2 cells and found no accumulation of R-loops at this locus (FIG. 10A). Finally, ChIP-qPCR using an anti-H3K9ac antibody showed that R-loop-positive loci from the DRIP experiment, namely the VGF, and HBA2 genes were hyperacetylated following treatment with the combination and to a lesser extent with romidepsin (FIG. 10B).

Histone Acetylation and Hypertranscription are Associated with Replication Fork Stalling and Increase in DNA Damage.

Having observed R-loop accumulation following treatment with the combination, it was hypothesized that hypertranscription, persistent hyperacetylation, and R-loop accumulation could increase the incidence of transcription replication conflicts, genome instability, and DNA damage and contribute to cell death. In response to DNA damage, replication protein A2 (RPA2) is phosphorylated at multiple sites by different protein kinases, including ATM, ATR, and DNA-PK, while the serine threonine-protein kinase, Chk1 (checkpoint kinase 1), is phosphorylated and activated by ATR. Accumulation of phospho-RPA2 was observed following treatment with the combination and single-agent romidepsin, and phospho-Chk1 primarily following treatment with the combination (FIG. 11A). Looking at replication fork dynamics in both MIA PaCa-2 and KLM cells, significant reduction in replication fork speed was observed following treatment, mainly with the combination and with single-agent romidepsin indicating stalling of replication forks. Drug treatments were for 24 hours (i.e., romidepsin treatment in combination or single agent romidepsin was 6 hours followed by 18-hour incubation with 2 nM DMPatA or with fresh medium respectively). Consistent with this in both MIA PaCa-2 and KLM cells, accumulation of DNA double stranded damage was detected following treatment with the combination by detecting the accumulation of phospho-histone H2A.X (γH2AX) protein over time alongside persistence of histone hyperacetylation (FIG. 11B).

Tolerability and Efficacy Studies of Treatment with the Combination in the Xenograft Model of PDAC.

Animal studies were performed according to Institutional Animal Care and Use Committee (IACUC) protocol number AC-AABD2601. Following the establishment of initial dose levels for individual drugs, efficacy of the combination and single agents in a MIA PaCa-2 xenograft model was assessed at a tolerable dose and schedule. A regression in tumor volume with single agent romidepsin or DMPatA and larger reductions with the combination was observed (FIG. 2A). Compared to vehicle, neither the single agent nor the combination treatments had a significant impact on weight confirming the safety of the tested doses and regiment of the combination and single drugs in vivo (FIG. 2B).

Additionally, snap-frozen tumor tissue samples from the mice treated with vehicle, single agents, and the combination were collected at the end of the study. These samples were sectioned, and the levels of H3K9ac and c-MYC proteins in tumor tissues were assessed by immunohistochemistry. In line with the in vitro observation of increased histone acetylation in the combination treated cells, levels of H3K9ac increased significantly in tumor from mice treated with romidepsin and the combination, and the levels of histone acetylation were significantly higher in the mice treated with the combination compared to the romidepsin monotherapy (FIG. 2C). Similarly, lower levels of c-MYC protein expression observed in the tumor samples from mice treated with DMPatA and the combination, in line with the in vitro observation that following treatment with DMPatA and the combination expression of c-MYC protein decreased in PDAC cell lines (FIG. 2D).

### Materials and Methods

### Reagents and Cell Culture

Romidepsin (depsipeptide, NSC 630176) was obtained from (Sigma-Aldrich #SML1175). Entinostat (#S1053) and chidamide (#S8567) were obtained from Selleckchem.com. Zotatifin (eFT226, #HY-112163) was obtained from MedChemExpress.com.

MIA PaCa-2, KLM, ASPC1, PANC-1, PANC 02.13, Capan-1, Cell lines was purchased from American Type Culture Collection. BXPC3 was a gift from Dr. Kenneth Olive, Columbia University. All cell lines were regularly authenticated by Genetic Resources Core Facility (GRCF) at Johns Hopkins University. Cells were cultured in appropriate cell culture medium (Gibco Laboratories, Grand Island, USA) supplemented with 10% fetal bovine serum (Gibco Laboratories, Grand Island, USA), and 1% glutamine (Gibco Laboratories, Grand Island, USA).

c-MYC-expressing MIA PaCa-2 cells were generated by transfecting wild type MIA PaCa-2 cells with 2 μg of pCDNA3mycIRESGFP DNA using Lipofectamine 2000 transfection reagent (Invitrogen, Cat #11668027). The pCDNA3mycIRESGFP plasmid was created by introducing the BamHI-XhoI fragment of pWZL Blast myc (Addgene plasmid #10674) containing the c-Myc cDNA in BamHI-XhoI restricted pcDNA3.1(+) IRES GFP (Life Technologies, Carlsbad, USA).

### Cell Viability and Annexin V Assay

Cell viability was determined using CellTiter-Glo® reagent (Promega, #G7570) following the manufacturer's instructions. Cells were seeded at 0.5×10⁶ cells in white 96-well plates and grown overnight. They were then treated and incubated at 37 °C with 5% CO₂. After incubation, assay reagent was added, and luminescent signals were quantified using a microplate reader (PHERAstar® FS, BMG LABTECH, Ortenberg, Germany). Caspase-Glo 3/7 assay (Promega, #G8091) was also employed following the manufacturer's instructions to measure apoptosis.

### RNA-Seq Gene Expression Analysis

Total RNA was extracted using RNeasy mini kit (Qiagen) from cells collected after 18 hours (6 hours romidepsin (15 nM)+12 hours washout with or without DMPatA (2 nM)) incubation with or without drugs. RNA quantitation and quality were assessed by the Agilent Bioanalyzer 2100 at the Molecular Pathology Shared Resource of the Herbert Irving Comprehensive Cancer Center at Columbia University (mBank). RNA library preparation and sequencing was performed at Columbia University's Sulzberger Genome Center. Briefly, poly-A pull-down was used to enrich mRNAs from total RNA samples, followed by library construction using Illumina TruSeq chemistry. Libraries were then sequenced using Illumina NovaSeq 6000. For the DESeq2 (v 1.40.2) analysis the data were imported with tximport in DESeq2 and a dds object was generated. Variance stabilizing transformation was used on the object and the output was used for the heatmap and PCA plots. Abundance of transcripts was estimated by pseudoalignment using kallisto, and downstream analysis was performed using R (v4.3.1) and the indicated packages. Low expression genes (inferior to 5 in more than 85% samples) were filtered out. Differentially expressed genes were determined using linear regression method (limma v3.56.2), and genes with |log 2FC|≥1 and adjusted p-value <0.05 were plotted accordingly on volcano plots and heatmap (ggplot2 v3.5.0 and ComplexHeatmap v2.16.0). Pathway enrichment was estimated by hypergeometric test (ClusterProfiler v4.8.3) using gene ontology biological process (GO BP) database, with a p-value adjustment by Benjamini-Hochberg method, and RNAseq filtered-in genes as gene universe. Pathways with an adjusted p-value <0.05 for each treatment were then concatenated together. Redundancy in filtered pathways was identified by semantic similarity (Wang distance method) using rrvgo (v1.12.2), and pathway number was reduced accordingly to 35 pathways. Genes associated to some common enriched pathways were represented on chord diagram (circlize v 0.4.16).

### Labeling of Nascent RNA by EU

To detect changes in the global RNA transcription we used the Click-iT RNA Alexa Fluor 594 Imaging Kit (Invitrogen, #C10330). Briefly, cells were cultured in complete media and pulsed for 60 min with alkyne-modified nucleoside, 5-ethynyl uridine (EU) at a final concentration of 1 mM. After fixation with 3.7% formaldehyde for 15 minutes and permeabilization with 0.1% Triton X-100, the 1× Click-iT reaction cocktail was applied for 30 minutes at room temperature. Following washing, DNA was stained with Hoechst 33342. Images were captured at 40× magnification using a Zeiss Confocal Microscope (Zeiss LSM 700), and Fiji (ImageJ, version: 1.54f, NIH, USA) software was used for image analysis.

### Western Blotting

Western blotting was carried out as previously described (Luchenko V L, Litman T, Chakraborty A R, et al. 65. Membranes were probed with primary antibodies: Phospho-Histone H2A.X (Ser139) (1:1000, Millipore Sigma #05-636), GAPDH (1:10000, Abcam #ab8245), c-Myc (1:1000, Abcam #ab32072), β-Actin (1:1000, Cell Signaling Technology (CST), #8457), Phospho-RPA32/RPA2 (Ser33) (1:1000, Cell Signaling Technology #10148), Phospho-Chk1 (Ser345) (1:1000, CST #2341), and secondary antibodies IRDye® 680RD Goat anti-Mouse and IRDye® 800CW Goat anti-Rabbit (both from LI-COR Biosciences). Signal quantification was performed using the Odyssey CLx imaging system (LI-COR Biosciences), capturing various exposures within the linear range with ImageStudio software V3.1 (LI-COR Biosciences).

### DNA-RNA Immunoprecipitation (DRIP)

DRIP assay was performed according to the method previously described (Sanz L A, Chédin F. High-resolution, strand-specific R-loop mapping via S9. 6-based DNA-RNA immunoprecipitation and high-throughput sequencing. Nature protocols. 2019; 14(6):1734). Briefly, following gentle extraction of the genomic DNA, cells were subjected to digestion using a cocktail of restriction enzymes, treated with or without RNaseH (New England Biolabs #M0297L). DNA-RNA hybrids were then immunoprecipitated from the digested genomic DNA using 59.6 antibody (Kerafast #ENH001). Quantitative PCR was performed at the R-loop positive loci VGF and HBA2 genes and R-loop negative locus SNRPN gene with the primers listed in Table 1.

### ChIP-qPCR

The chromatin immunoprecipitation (ChIP) assay was performed using the Zymo-Spin ChIP kit (Zymo Research, Irvine, CA #D5210) according to manufacturer's instructions. After treatment, cells were harvested, and chromatin was cross-linked with formaldehyde, then mechanically sheared by sonication on ice. Protein-DNA complexes were precipitated using control immunoglobulin G (normal Rabbit IgG, Cell Signaling Technology #2729) and anti-Acetyl-Histone 13 (Lys9) (C5B11) antibodies (1:50, Cell Signaling Technology #9649S). Subsequently, ChIP-DNA was eluted, reverse-cross-linked, and purified. Quantitative PCR was performed in technical triplicates using SsoAdvanced Universal SYBR Green Supermix (Bio-Rad, #1725272) and specific primers for VGF, HBA2, and SNRPN genes (see Table 1 for primer details).

TABLE 1
Primers used for qPCR validation of DRIP and ChIP.

| Type | Name | Sequence (5′ to 3′) |
|---|---|---|
| Positive-control locus | VGF F | TTCTTCCGCTTCCGCTTC (SEQ ID NO: 1) |
| Positive-control locus | VGF R | ATTGAGCTGTCCACCAAACT (SEQ ID NO: 2) |
| Positive-control locus | HBA2 F | ACAGAAGCCAGGAACTTGTC (SEQ ID NO: 3) |
| Positive-control locus | HBA2 R | CTTCTCTGCACAGCTCCTAAG (SEQ ID NO: 4) |
| Negative-control locus | SNRPN F | GCCAAATGAGTGAGGATGGT (SEQ ID NO: 5) |
| Negative-control locus | SNRPN R | TCCTCTCTGCCTGACTCCAT (SEQ ID NO: 6) |

### Metabolic Flux Analysis (Agilent Seahorse XF Assay)

Oxygen consumption rates (OCR) and extracellular acidification rates (ECAR) were measured using the XF96 Extracellular Flux analyzer (Seahorse Bioscience). Data were normalized by cell number using the CyQUANT Cell Proliferation Kit (Thermo Fisher Scientific). For OCR measurement, 20,000 cells were plated in XF96 cell culture microplates (Seahorse Biosciences) and treated with combination or single agents (for 6 and 12 hours). Afterward, growth media was replaced with bicarbonate-free assay media (XF assay medium, Seahorse Biosciences) and incubated at 37 °C for 1 hour in a CO₂-free incubator. OCR was measured using the XF96 Extracellular Flux Analyzer under basal conditions or following the addition of Oligomycin (1 μM), FCCP (0.5 μM), and Rotenone/Antimycin A (0.5 μM), following the manufacturer's protocol. For extracellular acidification rate (ECAR) measurement, 20,000 cells were plated in XF96 cell culture microplates (Seahorse Biosciences) in appropriate cell culture media. Cells were treated with combination or single agents (for 6 and 12 hours). After treatment, the growth media was replaced with bicarbonate-free assay media (XF assay medium, Seahorse Biosciences) and incubated at 37 °C for 1 hour in a C02-free incubator. PER was measured using an XF96 Extracellular Flux Analyzer (Seahorse Biosciences) under basal conditions and following the addition of glucose (10 mM), Oligomycin (1 μM), and the glucose analog 2-deoxyglucose, 2DG (50 mM), following the manufacturer's protocol.

### Quantification of Cellular Acetyl-CoA Levels

Five million cells were seeded in 150 mm cell culture dishes. Cells were treated with combination or single agents for a total of 18 h (6-+12-hours). Following treatment cells were harvested on ice, washed, and scraped out under liquid nitrogen. Prior to extraction an aliquot of the cell pellet was collected and subjected to protein quantification.

For Acetyl-CoA extraction, a 100 μL aliquot of the thawed pellet was spiked with ¹³C2-acetyl-CoA as an internal standard, extracted with 400 μL of methanol, vortexed, and centrifuged at 18.0×G for 5 minutes at 4 °C. The methanol supernatant (400 μL) was then dried under nitrogen at 35 °C and reconstituted in 100 μL of 10 mM ammonium bicarbonate (pH 9.5) for LC/MS analysis. Calibration curve standards were similarly prepared as the cell pellets. Acetyl-CoA quantification by LC/MS was performed on an Agilent 1290 Infinity UHPLC/6495 triple quadrupole mass spectrometer, using multiple reaction monitoring. Data processing employed Mass Hunter software (Agilent), and calibration curves (R2≥0.99) were fitted using either linear or quadratic models with 1/X or 1/X\hat{ }2 weighting.

### In Vivo Experiment

Animal experiments were performed in accordance with procedures approved by the IACUC AC-AABD2601, at the Columbia University Oncology Precision Therapeutics and Imaging Core (OPTIC). Athymic nude mice were obtained from Jackson Labs. Mice were implanted with 2.5×10⁶ Mia-PaCa-2 cells, injected subcutaneously into the right flank with 50% Matrigel. Once tumors reached 150-250 mm³ in volume, mice were randomized and enrolled into the study. Each group had a total of eight mice per treatment group. Treatment groups included: (1) Vehicle (Romidepsin+DMPatA), (2) Romidepsin 0.75 mg/kg, (3) DMPatA (MZ735) 0.075 mg/kg, and (4) Combination: Romidepsin 0.75 mg/kg+DMPatA 0.075 mg/kg. Mice were dosed once every four days intraperitoneally (IP), for 2 weeks initially followed by once every seven days dosing for an additional 5-6 weeks. Body weights and tumor measurements were recorded twice weekly. Formulations for DMPatA were prepared utilizing a solution comprising 10% ethanol, 10% Cremophor EL, and 80% 4% glucose in PBS. Initially, DMPatA was dissolved in ethanol, followed by the sequential addition of the remaining constituents of the vehicle formulation. Formulations for romidepsin were made using 5% ethanol and 5% propylene glycol, and 90% USP saline.

### Immunohistochemistry of Tumor Tissues

Frozen tumor tissues from the in vivo experiment were sectioned and stained at the Columbia University Molecular Pathology Shared Resource. Briefly, sections underwent three 5-min PBS washes and were blocked using 10% normal goat serum in PBS for 40 min. Primary antibodies were applied and incubated for 2 h at room temperature: Histone H3 (acetyl K9) (1:200, Abcam #ab10812) or C-MYC (1:150, Abcam #ab32072), each diluted in DAKO antibody diluent. After additional PBS washes, sections were incubated with secondary antibodies, Donkey anti-rabbit Alexa 594 (1:200, Invitrogen #A21207) or Donkey anti-mouse Alexa 594 (1:200, Invitrogen #A11032), for 1 h at room temperature. Following three more PBS washes, sections were mounted with DAPI medium. Slide Images were captured at 60× magnification using a Zeiss Confocal Microscope (Zeiss LSM 980). Image analysis was performed using Fiji (ImageJ) software (version: 1.54f, open-source image processing software, NIH, USA).

While illustrative embodiments have been illustrated and described, it will be appreciated that various changes can be made therein without departing from the spirit and scope of the invention.

1. A method for treating pancreatic cancer, comprising administering to a subject in need thereof a therapeutically effective amount DMPatA, or a pharmaceutically acceptable salt thereof, and a therapeutically effective amount Class 1 HDAC inhibitor selected from the group consisting of romidepsin, entinostat, and chidamide.

2. The method of claim 1, wherein the pancreatic cancer is pancreatic ductal adenocarcinoma.

3. A method for modulating the histone code with persistent H3K9 and H3K27 acetylation, reducing in the cellular acetyl-CoA levels, decreasing cellular levels of c-MYC protein and its targets, and/or impairing glycolysis and OXPHOS processes in a subject, comprising administering to a subject in need thereof an effective amount of DMPatA, or a pharmaceutically acceptable salt thereof, and a Class 1 HDAC inhibitor selected from the group consisting of romidepsin, entinostat, and chidamide.

4. The method of claim 1, wherein DMPatA, or a pharmaceutically acceptable salt thereof, and a Class 1 HDAC inhibitor selected from the group consisting of romidepsin, entinostat, and chidamide are administered intravenously, intraperitoneally, or orally.

5. The method of claim 1, wherein DMPatA, or a pharmaceutically acceptable salt thereof, and the Class 1 HDAC inhibitor selected from the group consisting of romidepsin, entinostat, and chidamide are administered at the same time.

6. The method of claim 1, wherein DMPatA, or a pharmaceutically acceptable salt thereof, and the Class 1 HDAC inhibitor selected from the group consisting of romidepsin, entinostat, and chidamide, are administered at different times.

7. The method of claim 1, wherein the Class 1 HDAC inhibitor is romidepsin,

8. The method of claim 1, wherein the Class 1 HDAC inhibitor is entinostat.

9. The method of claim 1, wherein the Class 1 HDAC inhibitor is chidamide.

10. The method of claim 1, wherein the subject is human.

11. A pharmaceutical composition, comprising DMPatA, or a pharmaceutically acceptable salt thereof, and a Class 1 HDAC inhibitor selected from the group consisting of romidepsin, entinostat, and chidamide, and a pharmaceutically acceptable carrier.

12-13. (canceled)

14. A method for diagnosing pancreatic cancer in a subject, comprising assessing the level of c-MYC protein expression in the subject.


SEQUENCE LISTING (6 sequences)

SEQ ID NO: 1 - DNA, 18 bp, synthetic construct
  source 1..18: mol_type = other DNA
ttcttccgcttccgcttc

SEQ ID NO: 2 - DNA, 20 bp, synthetic construct
  source 1..20: mol_type = other DNA
attgagctgtccaccaaact

SEQ ID NO: 3 - DNA, 20 bp, synthetic construct
  source 1..20: mol_type = other DNA
acagaagccaggaacttgtc

SEQ ID NO: 4 - DNA, 21 bp, synthetic construct
  source 1..21: mol_type = other DNA
cttctctgcacagctcctaag

SEQ ID NO: 5 - DNA, 20 bp, synthetic construct
  source 1..20: mol_type = other DNA
gccaaatgagtgaggatggt

SEQ ID NO: 6 - DNA, 20 bp, synthetic construct
  source 1..20: mol_type = other DNA
tcctctctgcctgactccat